(19) United States
(12) Patent Application Publication
(10) Pub. No.: US 2005/0158397 A1
(43) Pub. Date: Jul. 21, 2005

(54) Materials from bone marrow stromal cells for use in forming blood vessels and producing angiogenic and trophic factors

(76) Inventors: Michael Chopp, Southfield, MI (US); Yi Li, Canton, MI (US); Xiaoguang Chen, Madison Heights, MI (US)

Correspondence Address: MORGAN, LEWIS & BOCKIUS LLP, 1701 MARKET STREET, PHILADELPHIA, PA, 19103-2921, US

(21) Appl. No.: 10/502,723

(22) PCT Filed: Jan. 14, 2003
(86) PCT No.: PCT/US03/01129

Related U.S. Application Data
(60) Provisional application No. 60/348,955, filed on Jan. 14, 2002.

(51) Int. Cl. A61K 35/30; A61K 35/00; C12P 21/04

(52) U.S. Cl. 424/570; 424/115; 424/93.7; 435/70.3

(57) ABSTRACT

A therapeutic for use in inducing angiogenesis and vasculogenesis, the therapeutic including angiogenesis and vasculogenesis inducing factors isolated from stem cells in conjunction with a pharamaceutically acceptable cell therapy. A method of amplifying the production of angiogenesis and vasculogenesis inducing factors secreted by exposing stem cells to and co-culturing the stem cells with a compound for increasing the production of angiogenesis and vasculogenesis inducing factors. Angiogenesis and vasculogenesis inducing factors isolated and purified from stem cells for use in a therapy. A process for obtaining the angiogenesis and vasculogenesis inducing factors as set forth above, the process including the steps of isolating and purifying human mesenchymal stem cells from tissue prior to differentiation and then culture expanding the mesenchymal stem cells to produce a tool for neurological and musculoskeletal therapy. Isolated and culture expanded mesenchymal stem cells under the influence of a requisite compound, that are capable of differentiating and producing a desired cell phenotype needed for tissue repair.

### BACKGROUND OF THE INVENTION

1. Technical Field

The present invention relates to methods and compositions for use as therapeutics. More specifically, the present invention relates to the use therapeutic creation of angiogenesis and production of angiogenic and trophic factors.

2. Background Art

Stroke is the third most common cause of death in the adult population of the United States, and is a major cause of disability. Stroke occurs when a section of the brain becomes infarcted, resulting in death of brain tissue from interruption of cerebral blood supply. Cerebral infarcts associated with acute stroke cause sudden and dramatic neurological impairment. Other neurological diseases also result in the death of tissue and neurological impairment.

Pharmacological interventions have attempted to maximize the blood flow to stroke affected brain areas that might be able to survive, but clinical effectiveness has proven elusive. As stated in Harrison's Principles of Internal Medicine (9^{th} Ed., 1980, p. 1926), “despite experimental evidence that . . . [cerebral vasodilators] increase the cerebral blood flow, as measured by the nitrous oxide method, they have not proved beneficial in careful studies in human stroke cases at the stage of transient ischemic attacks, thrombosis-in-evolution, or in the established stroke. This is true of nicotinic acid, Priscoline, alcohol, papaverine, and inhalation of 5% carbon dioxide. In opposition to the use of these methods is the suggestion that vasodilators are harmful rather than beneficial, since by lowering the systemic blood pressure they reduce the intracranial anastomotic flow, or by dilating blood vessels in the normal parts of the brain they steal blood from the infarct.”

Additionally, diseases of the cardiovascular system are a leading worldwide cause of mortality and morbidity. For example, heart failure has been increasing in prevalence. Heart failure is characterized by an inability of the heart to deliver sufficient blood to the various organs of the body. Current estimates indicate that over 5 million Americans carry the diagnosis of heart failure with nearly 500,000 new cases diagnosed each year and 250,000 deaths per year attributed to this disease. Despite significant therapeutic accomplishments in the past two decades, heart failure continues to increase in incidence reaching epidemic proportions and representing a major economic burden in developed countries.

Heart failure is a clinical syndrome characterized by distinctive symptoms and signs resulting from disturbances in cardiac output or from increased venous pressure. Moreover, heart failure is a progressive disorder whereby the function of the heart continues to deteriorate over time despite the absence of adverse events. Due to heart failure, inadequate cardiac output results.

Generally, there are two types of heart failure. Right heart failure is the inability of the right side of the heart to pump venous blood into pulmonary circulation. A back up of fluid in the body occurs and results in swelling and edema. Left heart failure is the inability of the left side of the heart to pump blood into systemic circulation. Back up behind the left-ventricle then causes accumulation of fluid in the lungs.

The main resulting effect of heart failure is fluid congestion. If the heart becomes less efficient as a pump, the body attempts to compensate for it by, for example, using hormones and neural signals to increase blood volume.

Heart failure has numerous causes. For example, disease of heart tissue results in dead myocardial cells that no longer function. Progression in left ventricular dysfunction has been attributed, in part, to ongoing loss of these cardiomyocytes.

There have been numerous methods of treating and preventing heart failure. For example, stem cells have been used to regenerate cardiac cells in acute cardiac ischemia and/or infarction or injury in animal models. In one particular example, viable marrow stromal cells isolated from donor leg bones were culture-expanded, labeled, and then injected into the myocardium of isogenic adult rat recipients. After harvesting the hearts from 4 days to 12 weeks after implantation, the implantation sites were examined and it was found that implanted stromal cells showed the growth potential in a myocardial environment. (Wang, et. al.)

Cardiomyocytes have been shown to differentiate in vitro from pluripotent embryonic stem (ES) cells of line D3 via embryo-like aggregates (embryoid bodies). The cells were characterized by the whole-cell patch-clamp technique, morphology, and gene expression analogy during the entire differentiation period. (Maltsev, et. al., 1994) Additionally, pluripotent mouse ES cells were capable to differentiate into cardiomyocytes expressing major features of mammalian heart (Maltsev, et. al., 1993).

Stem cells regardless of their origin (embryonic, bone marrow, skeletal muscle, etc) have the potential to differentiate into various, if not all, cell types of the body. Stem cells are able to differentiate into functional cardiac myocytes. Thus, the development of stem cell-based therapies for treating heart failure has many advantages over exiting therapies.

### SUMMARY OF THE INVENTION

According to the present invention, there is provided a therapeutic for use in inducing angiogenesis and vasculogenesis. The therapeutic can include angiogenesis and vasculogenesis inducing factors isolated from stem cells in conjunction with a pharamaceutically acceptable cell therapeutic for inducing angiogenesis and vasculogenesis. Also provided is a method of amplifying the production of angiogenesis and vasculogenesis inducing factors secreted by exposing to and co-culturing stromal cells with a compound for increasing the production of the angiogenesis and vasculogenesis inducing factors. Angiogenesis and vasculogenesis inducing factors isolated and purified from stem cells for use in a therapy are also provided. There is provided a process for obtaining the angiogenesis and vasculogenesis inducing factors as set forth above, the process including the steps of isolating and purifying human mesenchymal stem cells from tissue prior to differentiation and then culture expanding the mesenchymal stem cells to produce a tool for neurological and musculoskeletal therapy. Isolated and culture expanded mesenchymal stem cells under the influence of a requisite compound, capable of differentiating and producing a desired cell phenotype needed for tissue repair are also provided.

### BRIEF DESCRIPTION OF THE FIGURES

Other advantages of the present invention are readily appreciated as the same becomes better understood by reference to the following detailed description when considered in connection with the accompanying drawings wherein:

FIGS. 1A through E are photographs showing the secretions of growth factors of BDNF (FIG. 1A), NGF (FIG. 1B), bFGF (FIG. 1C), VEGF (FIG. 1D) and HGF (FIG. 1E);

FIGS. 2A and B are graphs showing the results of behavioral function tests in rats before and after occlusion of the middle cerebral artery and treatment with intravenous MSC or no treatment;

FIGS. 3A and B are photographs showing the use of the rat corneal neovascularisation model to test whether MSC secretion induces angiogenesis in vivo, FIG. 3A shows an sham-operated cornea with no evidence of neovascularisation and FIG. 3B shows MSC supernatant placed in collagen water inserted within the corneal pocket wherein robust corneal neovascularisation is evident;

FIG. 4 shows an illustration of the experiments performed to support the present invention, wherein bone marrow is extracted from an animal and the MSC are separated and cultured in three to five passages, the MSC are injected into an animal with neural injury and the cells migrate selectively to injured tissue and localize to the boundary zone of the lesion, the MSC then activate an array of restorative events that are mediated by MSC, parenchymal-cell secretions and growth and trophic factors, thus improving neurological function;

FIG. 5 shows a standard coronal section identified at the level of the cnterior commisure of rat brain that divides the right hemisphere into three subregions and eight fields;

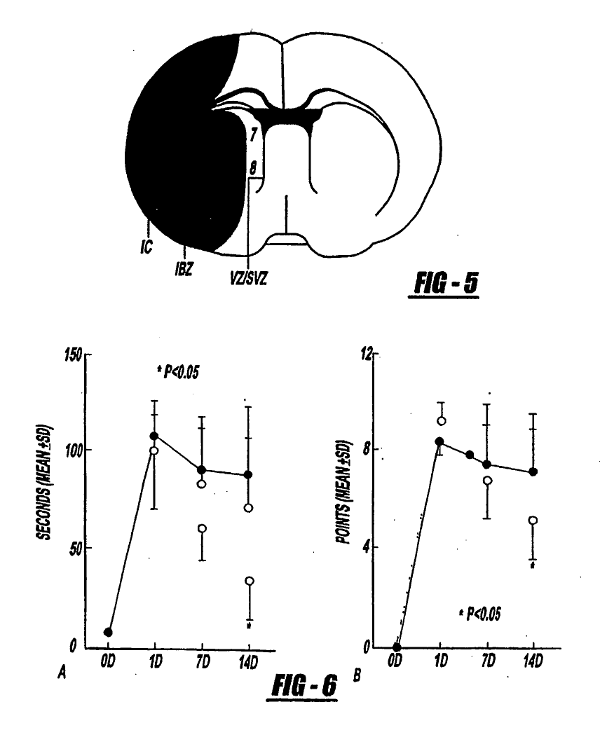

FIGS. 6A and B are graphs that show the results of behavioral function tests before and after middle cerebral artery occlusion;

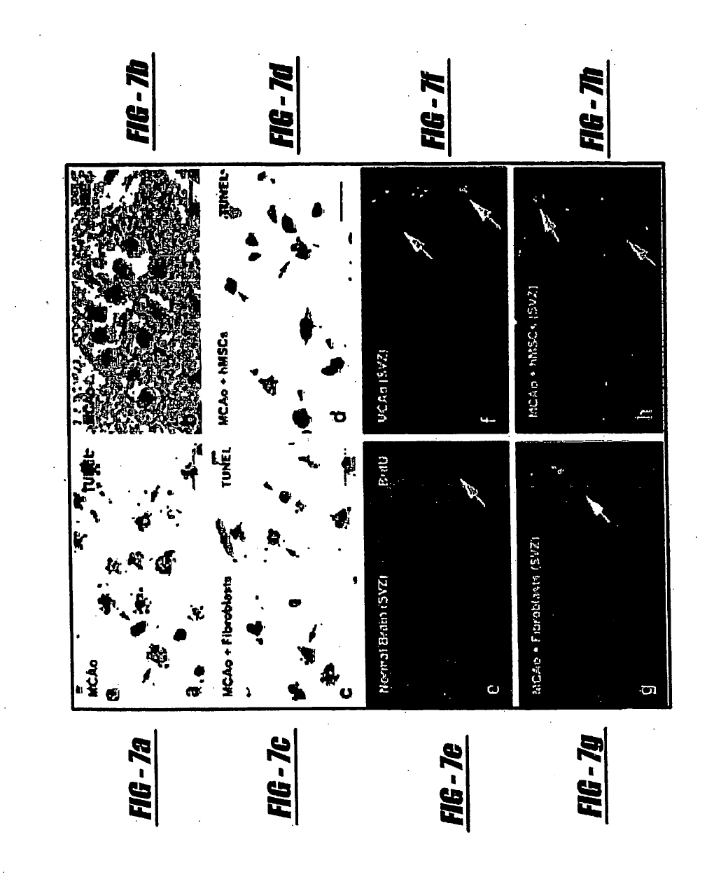

FIGS. 8A and B are graphs showing a mixed lymphocyte reaction between rat spleen cells and hMSC; and

FIG. 9 photomicrographs showing the morphologic characteristics of exogenous human bone marrow stromal cells (hMSC) and endogenous brain cells in rat brain.

### DESCRIPTION OF THE INVENTION

Generally, the present invention provides for the use of angiogenesis and vasculogenesis inducing factors from bone marrow stromal cells or other stem cells as part of cell therapy for inducing angiogenesis and vasculogenesis. More specifically, the present invention provides a method of amplifying the production of the angiogenesis and vasculogenesis inducing factors (e.g. angiogenic, trophic, and growth factors) secreted by stromal or other stem cells for use in a therapy, the factors inducing angiogenesis, or other beneficial growth, upon administration. This amplification occurs with exposure to and co-culture of the cells with brain extract and/or with calcium.

The term “angiogenesis” is defined as a process of tissue vascularization that involves the growth of new and/or developing blood vessels into a tissue, and is also referred to as neo-vascularization. The process is mediated by the infiltration of endothelial cells and smooth muscle cells. The process can proceed in one of three ways: the vessels can sprout from pre-existing vessels, de-novo development of vessels can arise from precursor cells (vasculogenesis), and/or existing small vessels can enlarge in diameter.

The terms “enhance” or “enhancement” as used herein are meant to include, but are not limited to, making rich or richer by the addition or increase of some desirable quality or quantity of substance.

The phrase “brain extract” as used herein is meant to include, but is not limited to, brain cells or other similar cells obtained from the brain. These cells can also be cultured with a medium and the supernatant can be used as a brain extract.

The term “injury”, as used herein, is intended to include, but is not limited to, physical or biological injuries including genetic disorders, diseases, and age onset disorders. For example, patients suffer neurological and functional deficits after stroke, CNS injury, and neurodegenerative disease.

The term “cell therapy” as used herein includes, but is not limited to, the therapeutic use of stem cells. A stem cell is a generalized mother cell whose descendants specialize into various cell types. Stem cells have various origins including, but not limited to, embryo, bone marrow, liver, stromal, fat tissue, and other stem cell origins known to those of skill in the art. These stem cells can be placed into desired areas as they naturally occur, or can be engineered in any manner known to those of skill in the art. Thus, through various genetic engineering methods including, but not limited to, transfection, deletion, and the like, stem cells can be engineered in order to increase their likelihood of survival or for any other desired purpose.

Stem cells are capable of self-regeneration when provided to a human subject in vivo, and can become lineage-restricted progenitors, which further differentiate and expand into specific lineages. As used herein, “stem cells” refers to human marrow stromal cells and not stem cells of other cell types. Preferably, “stem cells” refers to human marrow stromal cells.

The term “stem cell” or “pluripotent” stem cell are used interchangeably to mean a stem cell having (1) the ability to give rise to progeny in all defined hematopoietic lineages, and (2) stem cells capable of fully reconstituting a seriously immunocompromised host in all blood cell types and their progeny, including the pluripotent hematopoietic stem cell, by self-renewal.

Bone marrow is the soft tissue occupying the medullary cavities of long bones, some haversian canals, and spaces between trabeculae of cancellous or spongy bone. Bone marrow is of two types: red, which is found in all bones in early life and in restricted locations in adulthood (i.e. in the spongy bone) and is concerned with the production of blood cells (i.e. hematopoiesis) and hemoglobin (thus, the red color); and yellow, which consists largely of fat cells (thus, the yellow color) and connective tissue.

As a whole, bone marrow is a complex tissue including hematopoietic stem cells, red and white blood cells and their precursors, mesenchymal stem cells, stromal cells and their precursors, and a group of cells including fibroblasts, reticulocytes, adipocytes, and endothelial cells which form a connective tissue network called “stroma”. Cells from the stroma morphologically regulate the differentiation of hematopoietic cells through direct interaction via cell surface proteins and the secretion of growth factors and are involved in the foundation and support of the bone structure.

Studies using animal models have suggested that bone marrow contains “pre-stromal” cells that have the capacity to differentiate into cartilage, bone, and other connective tissue cells. (Beresford, J. N.: Osteogenic Stem Cells and the Stromal System of Bone and Marrow, Clin. Orthop., 240:270, 1989). Recent evidence indicates that these cells, called pluripotent stromal stem cells or mesenchymal stem cells, have the ability to generate into several different types of cell lines (i.e. osteocytes, chondrocytes, adipocytes, etc.) upon activation. However, the mesenchymal stem cells are present in the tissue in very minute amounts with a wide variety of other cells (i.e. erythrocytes, platelets, neutrophils, lymphocytes, monocytes, eosinophils, basophils, adipocytes, etc.), and, in an inverse relationship with age, they are capable of differentiating into an assortment of connective tissues depending upon the influence of a number of bioactive factors.

The purpose of the present invention is to utilize bone marrow stromal cells, supernatant from bone marrow stromal cells, or the secretions resulting from the interaction of bone marrow stromal cells and other stem cells for the treatment of disease. These secretions include, but are not limited to, an array of growth, trophic, and angiogenesis factors. The method of the present invention promotes an improved outcome for the recovery from neuronal injury, or other injury, by augmenting the effects of the treatment, for example angiogenesis, and augmenting the blood vessel production formed from the non-existing or pre-existing vasculature. The present invention can also be used to provide means to enhance brain compensatory mechanism to improve function after CNS damage or degeneration. Additionally, the methods and compositions of the present invention can enhance the effectiveness of cell therapy.

Enriching and/or repopulating the injured cells through transplanted stem cells that differentiate into the injured cells increase function. For example, when this therapy is used in the heart the therapy can increase the contractile units in the heart. The increase of contractile units increases the function of the heart. Additionally, the stem cells can also be responsible for the release of various substances such as trophic factors. Thus, for example, the release of trophic factors induces angiogenesis (increase of the number of blood vessels) in order to increase cardiac function and/or treat heart failure. Therefore, the stem cells operate to increase cardiac function and/or treat heart failure through various mechanisms other than just differentiating into functional cardiac muscle cells.

The production of trophic factors, growth factors, and angiogenic factors is typically an expensive and difficult process. The method and composition of the present invention provide an inexpensive and simple method of producing pure trophic factors, growth factors, and other related factors merely by administering the therapy of the present invention. These factors can be used for treatment patients. For example, the factors can be used for inducing angiogenesis, vasculogenesis, and to enhance function and repair of tissues both in vivo and in vitro. It is therefore beneficial to determine what bone marrow stromal cells can be employed as cellular factories for producing and secreting trophic, growth, and angiogenic factors. These factors can include, but are not limited to, VEGF, HGF, BDNF, NGF, bFGF, etc. The methods of the present invention allow the production of these factors to be manipulated by culture conditions. For example culture conditions can be manipulated by co-culturing cells with tissue and/or different calcium concentrations in the culture medium.

The present invention is based on the use of cell therapy to treat disease. Although stem cells have different origins (embryo, bone marrow, liver, fat tissue, etc.), their important common characteristic is that they have the potential to differentiate into various, if not all, cell types of the body. As previously mentioned, stem cells have been shown to be able to differentiate into cardiac muscle cells. (Maltsev et al., 1993; 1994).

Applicants have developed a process for isolating and purifying human mesenchymal stem cells from tissue prior to differentiation and then culture expanding the mesenchymal stem cells to produce a valuable tool for neurological and musculoskeletal therapy. The objective of such manipulation is to greatly increase the number of mesenchymal stem cells and to utilize these cells to redirect and/or reinforce the body's normal reparative capacity. The mesenchymal stem cells are harvested in great numbers and applied to areas of tissue damage to enhance or stimulate in vivo growth for regeneration and/or repair, to improve implant adhesion to various prosthetic devices through subsequent activation and differentiation, enhance hemopoietic cell production, etc.

Various procedures are contemplated by the inventors for transferring, immobilizing, and activating the culture expanded, purified mesenchymal stem cells at the site for repair, implantation, etc., including injecting the cells at the site of a skeletal defect, incubating the cells with a prosthesis and implanting the prosthesis, etc. Thus, by isolating, purifying and greatly expanding the number of cells prior to differentiation and then actively controlling the differentiation process by virtue of their positioning at the site of tissue damage or by pretreating in vitro prior to their transplantation, the culture-expanded, undifferentiated mesenchymal stem cells can be utilized for various therapeutic purposes such as to elucidate cellular, molecular, and genetic disorders in a wide number of neurologic diseases, neural injury, metabolic bone diseases, skeletal dysplasias, cartilage defects, ligament and tendon injuries and other musculoskeletal and connective tissue disorders.

Various procedures are contemplated by the inventors for transferring, immobilizing, and activating the mesenchymal stem or progenitor cells at the site for repair, implantation, etc., through the use of various porous ceramic vehicles or carriers, including injecting the cells into the location of injury.

The human mesenchymal stem cells can be obtained from a number of different sources, including plugs of femoral head cancellous bone pieces, obtained from patients with degenerative joint disease during hip or knee replacement surgery, and from aspirated marrow obtained from normal donors and oncology patients who have marrow harvested for future bone marrow transplantation. Although the harvested marrow was prepared for cell culture separation by a number of different mechanical isolation processes depending upon the source of the harvested marrow (i.e. the presence of bone chips, peripheral blood, etc.), the critical step involved in the isolation processes was the use of a specially prepared medium that contained agents which allowed for not only mesenchymal stem cell growth without differentiation, but also for the direct adherence of only the mesenchymal stem cells to the plastic or glass surface area of the culture dish. By producing a medium that allowed for the selective attachment of the desired mesenchymal stem cells that were present in the marrow samples in very minute amounts, it was possible to separate the mesenchymal stem cells from the other cells (i.e. red and white blood cells, other differentiated mesenchymal cells, etc.) present in the bone marrow.

As indicated above, the complete medium can be utilized in a number of different isolation processes depending upon the specific type of initial harvesting processes used in order to prepare the harvested bone marrow for cell culture separation. When plugs of cancellous bone marrow were utilized, the marrow was added to the complete medium and vortexed to form a dispersion which was then centrifuged to separate the marrow cells from bone pieces, etc. The marrow cells (consisting predominantly of red and white blood cells, and a very minute amount of mesenchymal stem cells, etc.) were then dissociated into single cells by passing the complete medium containing the marrow cells through syringes fitted with a series of 16, 18, and 20 gauge needles. It is believed that the advantage produced through the utilization of the mechanical separation process, as opposed to any enzymatic separation process, was that the mechanical process produced little cellular change while an enzymatic process could produce cellular damage particularly to the protein binding sites needed for culture adherence and selective separation, and/or to the protein sites needed for the production of monoclonal antibodies specific for said mesenchymal stem cells. The single cell suspension (which was made up of approximately 50-100×10⁶ nucleated cells) was then subsequently plated in 100 mm dishes for the purpose of selectively separating and/or isolating the mesenchymal stem cells from the remaining cells found in the suspension.

When aspirated marrow was utilized as the source of the human mesenchymal stem cells, the marrow stern cells (which contained little or no bone chips but a great deal of blood) were added to the complete medium and fractionated with Percoll (Sigma, St. Louis, Mo.) gradients more particularly described below. The Percoll gradients separated a large percentage of the red blood cells and the mononucleate hematopoietic cells from the low-density platelet fraction that contained the marrow-derived mesenchymal stem cells. The platelet fraction, which contained approximately 30-50×10⁶ cells, was made up of an undetermined amount of platelet cells, 30-50×10⁶ nucleated cells, and only about 50-500 mesenchymal stem cells depending upon the age of the marrow donor. The low-density platelet fraction was then plated in the Petri dish for selective separation based upon cell adherence.

The marrow cells obtained from either the cancellous bone or iliac aspirate (i.e. the primary cultures) were grown in complete medium and allowed to adhere to the surface of the Petri dishes for one to seven days according to the conditions set forth below. Since no increase in cell attachment was observed after the third day, three days was chosen as the standard length of time at which the non-adherent cells were removed from the cultures by replacing the original complete medium with fresh complete medium. Subsequent medium changes were performed every four days until the culture dishes became confluent which normally required 14-21 days. This represented a 10³-10⁴-fold increase in undifferentiated human mesenchymal stem cells.

The cells were then detached from the culture dishes utilizing a releasing agent such as trypsin with EDTA (ethylene diaminetetra-acetic acid) (0.25% trysin, 1 mM EDTA (1.times.), Gibco, Grand Island, N.Y.) or a chelating agent such as EGTA (ethylene glycol-bis-(2-amino ethyl ether) N,N′-tetraacetic acid, Sigma Chemical Co., St. Louis, Mo.). The advantage produced through the use of a chelating agent over trypsin was that trypsin could possibly cleave off a number of the binding proteins of the mesenchymal stem cells. Since these binding proteins contain recognition sites, when monoclonal antibodies were sought to be produced, a chelating agent such as EGTA as opposed to trypsin, was utilized as the releasing agent. The releasing agent was then inactivated and the detached cultured undifferentiated mesenchymal stem cells were washed with complete medium for subsequent use.

Under certain conditions, culture expanded mesenchymal stem cells have the ability to differentiate into bone when incubated as a graft in porous calcium phosphate ceramics. Although the internal factors which influence the mesenchymal stem cells to differentiate into bone as opposed to cartilage cells are not well known, it appears that the direct accessibility of the mesenchymal stem cells to growth and nutrient factors supplied by the vasculature in porous calcium phosphate ceramics, as opposed to the diffusion chamber, influenced the differentiation of the mesenchymal stem cells into bone. Further, the brain extract causes the stem cells to create additional trophic factors further enhancing the effect of the stem cells.

As a result, the isolated and culture expanded mesenchymal stem cells can be utilized under certain specific conditions and/or under the influence of certain factors, to differentiate and produce the desired cell phenotype needed for tissue repair.

Administration of a single dose of mesenchymal stem cells can be effective to reduce or eliminate the T cell response to tissue allogeneic to the T cells or to “non-self” tissue, particularly in the case where the T lymphocytes retain their non-responsive character (i.e., tolerance or anergy) to allogeneic cells after being separated from the mesenchymal stem cells.

The general method of transplanting stem cells with brain extract into the myocardium occurs by the following procedure. The stem cells and the brain extract are administered to the patient. The administration can be subcutaneously, parenterally including intravenous, intraarterial, intramuscular, intraperitoneally, and intranasal administration as well as with intrathecal and infusion techniques.

The dosage of the mesenchymal stem cells varies within wide limits and is fitted to the individual requirements in each particular case. In general, in the case of parenteral administration, it is customary to administer from about 0.01 to about 5 million cells per kilogram of recipient body weight. The number of cells used will depend on the weight and condition of the recipient, the number of or frequency of administrations, and other variables known to those of skill in the art. The mesenchymal stem cells can be administered by a route that is suitable for the tissue, organ, or cells to be transplanted. They can be administered systemically, i.e., parenterally, by intravenous injection or can be targeted to a particular tissue or organ, such as bone marrow. The human mesenchymal stem cells can be administered via a subcutaneous implantation of cells or by injection of stem cell into connective tissue, for example muscle.

The cells can be suspended in an appropriate diluent, at a concentration of from about 0.01 to about 5×10⁶ cells/ml. Suitable excipients for injection solutions are those that are biologically and physiologically compatible with the cells and with the recipient, such as buffered saline solution or other suitable excipients. The composition for administration must be formulated, produced and stored according to standard methods complying with proper sterility and stability.

Although the invention is not limited thereto, mesenchymal stem cells can be isolated, preferably from bone marrow, purified, and expanded in culture, i.e. in vitro, to obtain sufficient numbers of cells for use in the methods described herein. Mesenchymal stem cells, the formative pluripotent blast cells found in the bone, are normally present at very low frequencies in bone marrow (1:100,000) and other mesenchymal tissues. See, Caplan and Haynesworth, U.S. Pat. No. 5,486,359. Gene transduction of mesenchymal stem cells is disclosed in Gerson et al U.S. Pat. No. 5,591,625.

Unless otherwise stated, genetic manipulations are performed as described in Sambrook and Maniatis, MOLECULAR CLONING: A LABORATORY MANUAL, 2nd Ed.; Cold Spring Harbor Laboratory Press, Cold Spring Harbor, N.Y. (1989).

The present invention is valuable because it has become abundantly clear that one mechanism for the deterioration of function in heart failure of any etiology is due, in part, to the ongoing death of heart muscle cells (Sabbah, 2000). The solution to this problem is to enrich and/or repopulate the myocardium with new cardiac cells which take the place of lost cells or provide additional reinforcement of the currently functioning cardiac cells, thereby improving the pumping function of the failing heart.

The present invention is advantageous over all currently existing treatments because there are no known side effects and the treatment is relatively non-invasive. For example, treatment of heart failure is currently based primarily on the use of drugs that interfere with neurohumoral systems. Additionally, surgical treatment exists that include heart transplantation as well as the use of ventricular or bi-ventricular assisting devices. The advantages offered by the present invention is the ability to treat heart failure by directly addressing the primary cause of the disease, namely, loss of contractile units. Re-population of the myocardium with stem cells that differentiate into contractile units that contribute to the overall function of the failing heart, therefore, is novel and goes to the center of the problem. Other advantages include absence of side effects that are often associated with the use of pharmacological therapy and absence of immune rejection that plagues heart transplantation or other organ transplants and the ability to increase the trophic factors created by the stem cells.

The present invention has the potential to replace many current surgical therapies and possibly even pharmacological therapies. Devices currently exist that allow delivery of stem cells in conjunction with brain extract to the failing heart using catheter-based approaches, thus eliminating the need for open chest surgery. Additionally, the present invention is applicable in both the human medical environment and veterinary setting.

The method and composition of the present invention are exemplified in the Examples included herein. While specific embodiments are disclosed herein, they are not exhaustive and can include other suitable designs that vary in design and methodologies known to those of skill in the art. Basically, any differing design, methods, structures, and materials known to those skilled in the art can be utilized without departing from the spirit of the present invention.

### EXAMPLES

Methods:

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

General methods in immunology: Standard methods in immunology known in the art and not specifically described are generally followed as in Stites et al. (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Delivery of Therapeutics:

The cells of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the cells of the present invention can be administered in various ways. It should be noted that it can be administered as the cells or as pharmaceutically acceptable salt and can be administered alone or as an active ingredient in combination with pharmaceutically acceptable carriers, diluents, adjuvants and vehicles. The cells can be administered orally, subcutaneously or parenterally including intravenous, intraarterial, intramuscular, intraperitoneally, and intranasal administration as well as intrathecal and infusion techniques. Implants of the cells are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the cells of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for cells compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

Sterile injectable solutions can be prepared by incorporating the cells utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the cells utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the cells utilized in the present invention can be administered orally to the patient. Conventional methods such as administering the cells in tablets, suspensions, solutions, emulsions, capsules, powders, syrups and the like are usable. Known techniques that deliver it orally or intravenously and retain the biological activity are preferred.

In one embodiment, the cells of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated and will vary from about 100 ng/kg of body weight to 100 mg/kg of body weight per day and preferably will be from 10 mg/kg to 10 mg/kg per day.

### Example 1

Treatment of traumatic brain injury (TBI) with bone marrow stromal cells (MSCs) improves functional outcome in rat. Tissue replacement is not the only compensatory avenue in cell transplantation therapy. As various growth factors have been shown to mediate the repair and replacement of damaged tissue, MSCs provide trophic support that plays a role in the treatment of damaged tissue. The response of human MSCs (hMSCs) to the cerebral tissue extract from TBI was investigated and tested to determine whether the TBI environment induces hMSC differentiation and growth factor secretion. hMSCs were cultured with TBI extracts in vitro and immunocytochemistry and quantitative sandwich enzyme-linked immunosorbent assay (ELISA) were performed. The results show that TBI conditioned hMSCs expressed specific cellular protein markers: NeuN for neuronal nuclear (0.2-0.5% of total hMSCs)., Tuj-1 for early neuronal differentiation and neurite outgrowth (6-10%), GFAP for astrocyte (4-7%) and MBP for oligodendrocyte (3-5%). In addition, hMSCs treated with TBI extracts respond by up-regulating the secretions of brain-derived neurotrophic factor (BDNF), nerve growth factor (NGF), basic fibroblast growth factor (bFGF), vascular endothelial growth factor (VEGF) and hepatocyte growth factor (HGF) in a time-dependent manner. These data demonstrate that TBI extracts drive hMSCs to express neural morphology and proteins phenotypic of the brain tissue. Furthermore, the ELISA data shows that transplanted hMSCs provide therapeutic benefit via a responsive secretion of an array of growth factors that can foster neuroprotection and angiogenesis.

Bone marrow stromal cells (MSCs), when transplanted intravenously into rats subjected to traumatic brain injury (TBI), promote neurological functional recovery (Lu et al., 2001 a). Upon transplantation, MSCs migrate preferentially to the locale of compromised tissue, and some cells express proteins phenotypic of brain endogenous-like cells (Lu et al., 2001b; Lu et al., 2001a; Mahmood et al., 2001). Although the long-term strategy of replacement of injured tissue by a stem cell population is a straightforward approach to the treatment of neural injury, the low level of MSC differentiation in acute and short-term therapeutic transplantation of the TBI model, unlikely provides the functional benefit (Lu et al., 2001b; Lu et al., 2001a; Mahmood et al., 2001), and the mechanisms providing benefit remain unknown.

MSCs naturally produce a variety of cytokines and growth factors (Takai et al., 1997; Labouyrie et al., 1999; Bjorklund and Lindvall, 2000; Dormady et al., 2001), the secretive properties of which are influenced by their microenvironment (Dormady et al., 2001). Neurotrophins such as brain-derived neurotrophic factor (BDNF) and nerve growth factor (NGF) increase survival of injured CNS tissue both in vivo and in vitro (Hefti, 1986; Kromer, 1987; Koliatsos et al., 1993; Bullock et al., 1999; Gage, 2000). A widely studied growth factor in preclinical studies is basic fibroblast growth factor (bFGF) (Ay et al., 1999). bFGF administered intravenously within hours after the onset of ischemia reduces infarct size, presumably due to direct protection of cells at the borders (penumbra) of cerebral infarction (Ay et al., 1999). Vascular endothelial growth factor (VEGF) expression also promotes angiogenesis and neural repair (Papavassiliou et al., 1997). Treatment of stroke with VEGF improves functional outcome (Zhang et al., 2000b). Hepatocyte growth factor (HGF) expression is naturally up-regulated within the brain post-injury and displays anti-apoptotic effects on cerebral neurons in vitro (Zhang et al., 2000a).

Materials and Methods

Reagents

Hank's balanced salt solution (HBSS), Dulbecco's modified Eagle's medium (DMEM), Knockout DMEM, Knockout serum replacement, fetal bovine serum (FBS), trypsin and ethylenediamine-tetra acetic acid (EDTA) were purchased from GIBCO (Grand Island, N.Y.). Ficoll was purchased from Pharmacia (Piscataway, N.J.). Antibodies against monoclonal neuronal nuclear antigen (NeuN), polyclonal β-tubulin isotype1 (Tuj-1), glial fibrillary acid protein (GFAP) and myelin basic protein (MBP) were purchased from CHEMICON (Temecula, Calif.). Kits of sandwich enzyme-linked immunosorbent assays (ELISA) for BDNF, bFGF, VEGF and HGF were obtained from R & D systems (Minneapolis, Minn.). The NGF ELISA kit was made in the laboratory. Anti-β(2.5S, 7S). NGF monoclonal antibody, anti-β(2.5S, 7S) NGF-β-gal, NGF-β standard were purchased from Roche Molecular Biochemicals (Indianapolis, Ind.). Unless otherwise indicated, reagents were obtained from Sigma Chemical Co. (St. Louis, Mo.).

Primary hMSCs Culture

The primary bone marrow was obtained from 15-16 ml aspirates from the iliac crest of three normal human donors. Each aspirate was diluted 1:1 with HBSS and layered over about 10 ml of Ficoll. After centrifugation at 2,500×g for 30 minutes, the mononuclear cell layer was removed from the interface and suspended in HBSS. Cells were centrifuged at 1,000×g for 10 minutes and 5×10⁶ cells were resuspended to each 100-mm tissue culture dish (Falcon, Becton-Dickinson, N.J.) in complete DMEM supplemented with 10% FBS. The cells were incubated at 37° C. in 5% CO₂ in flasks for 3 days and nonadherent cells were removed by replacing the medium. After the cultures reached confluency, usually at 2-3 weeks, the cells were harvested with 0.05% w/v trypsin and 0.02% w/v EDTA in phosphate-buffered saline (PBS, pH 7.4) for 5 minutes at 37° C., replated and once again cultured for 2 weeks and harvested. The cells were then frozen for later use. Cells used in these experiments were harvested from 3 to 5 passages.

Extracts from Traumatic Injured Brain

Experiments were performed on male Wistar rats weighing 250 to 350 g (n=21). Anesthesia was induced in the rats by intraperitoneal administration of chloral hydrate (35 mg/100 g body weight). Rectal temperature was maintained at 37° C. throughout the surgical procedure using a feedback-regulated water heating system. Rats were placed in a stereotaxic frame. Injury was induced by impacting the left cortex (ipsilateral cortex) with a pneumatic piston having a 6 mm diameter tip at a rate of 4 m/second and 2.5 mm of compression (Dixon et al., 1991). Control animals underwent craniotomy, but received no injury. Rats were sacrificed at 1, 4 and 7 days (n=6 per time point) after operation. Brain tissue extracts were immediately obtained from the experimental and normal control (n=3) rats. Segments of the left hemisphere of both experimental rats and control rats were placed on ice, and the wet weight in grams was rapidly measured. Subsequently, the tissue pieces were homogenized by adding DMEM (150 mg tissue/ml DMEM) and were incubated on ice for 10 minutes. The homogenate was centrifuged for 10 minutes at 10,000×g at 4° C. The supernatant was collected and stored −80° C. for treatment of hMSCs.

Cell Differentiation

The protein phenotypic studies were performed by seeding 1.0×10⁶ cells in a 35-mm dish and treating them with fresh knockout DMEM with 20% knockout serum replacement containing 10%, 20% or 40% of TBI tissue extract supernatant. All cells were incubated for 7 days. Estimates of immunoreactive neural-like cells were based on counting cells in 10 random visual fields (10× objective) in three culture dishes in a minimum of three different experiments. Percentages of phenotypic neural cells were calculated from the total number of cells.

Double and Triple Staining Immunocytochemistry

hMSCs were plated at a density of 1.0×10⁶ on glass cover slips (18×18 mm²) in 35-mm dishes using different treatments noted above. The cells on glass cover slips were used for immunocytochemistry. The supernatant of the culture medium was used for quantitative ELISA measurement as described below. The cells were washed with PBS (pH 7.4) and fixed with 4% paraformaldehyde for 10 minutes. Nonspecific binding sites were blocked with 4% normal horse serum, 2% bovine serum albumin and 0.1% Triton X-100 for 1 hour. The cover slips were washed with PBS and incubated with primary antibodies against Tuj-1, GFAP or MBP for 1 hour. They were washed again with PBS and incubated with fluorescein-isothiocynate (FITC) conjugated goat anti-mouse or anti-rabbit IgG secondary antibody for 1 hour. The Tuj-1 stained hMSC cover slips were once again washed and incubated with second primary antibody NeuN overnight, then washed with PBS and incubated with cyanine-5.18 (Cy5) conjugated anti-mouse IgG secondary antibody for 1 hour. 4′ b-Diamidine-2-phenylindole dihydrochloride (DAPI) dye was used to determine the number of cells by counting the nuclei in the field. The cover slips were then mounted with glycergel mounting medium.

ELISA

ELISA was used to measure the secretion of BDNF, NGF, bFGF, VEGF and HGF by hMSCs at 1, 4, and 7 days in culture conditioned by TBI and normal brain extract supernatant. In brief, all reagents and working standards were prepared as directed by the manufacturer, and 50-150 μl of standard or assay diluent solution was added per well in the 96 well plates. The wells were gently mixed, and incubated for 2-4 hours at room temperature. Each well was aspirated and washed, repeating the process three times. After the last wash, any remaining buffer was removed by aspirating or decanting the well, and 200 μl of various growth factor conjugates were added to each well. The plate was then incubated for 2-4 hours at room temperature. Aspiration and washing were repeated. 200 μl of substrate solution was added to each well and incubated for 15-30 minutes at room temperature. 50 μl of stop solution was added and gently mixed. The optical density of each well was determined within 30 minutes using a microplate reader set to 450-620 nm.

Statistical Analysis

Student's t-test was used to evaluate morphological differences between the stimulated samples and their respective control. The significance of time responses was assessed by repeated measures analysis of variance (ANOVA). The ELISA data were linearized by plotting the log of the various growth factor concentrations versus the log of the optical density, and best-fit line was determined by regression analysis. Average duplicate readings were made for each standard, control, and sample and the average zero standard optical density was subtracted. All values are expressed as mean±SD. p<0.05 was considered statistically significant.

Results

Morphological Differentiation of hMSCs into Neural-Like Cells

Phase contrast microscopy shows the normal morphology of fibroblast-like hMSCs cultured in complete DMEM supplemented with 10% FBS. After 7 days of exposure, in the knockout DMEM with 20% knockout serum replacement, some refractive cells exhibited short processes. A few (˜2-3% of total cells, Table 1) cells exhibited neuronal-like morphology in hMSCs cultured in normal brain tissue extract supernatant. However, normal brain extracts induced hMSC proliferation (1.56×10⁴±0.2×10⁴/ml) compared with hMSCs cultured in the knockout DMEM with 20% knockout serum replacement (1.24×10⁴±0.5×10⁴/ml) (p<0.05). Diverse morphology, but typically refractive cells with long branching processes (process length>10 μm) and growth cone—like terminal structures (˜13-30% neuron-like cells of total cells, Table), and stellate cells with small and multipolar processes were detected in hMSCs cultured in 20%˜40% TBI extract supernatant. There was a trend for the total numbers of cells in the TBI (1.08×10⁴±0.3×10⁴/ml) extract cultures to decrease, but this did not reach statistical significance. All of the various concentrations of TBI tissue extracts induced hMSCs to morphologically resemble neural-like cells.

Expression of Neural Markers by hMSCs

After 7 days in knockout DMEM with 20% knockout serum replacement and containing 10%, 20% or 40% of TBI tissue extract culture, hMSCs were processed for immunocytofluorescence. This permitted double labeling with DAPI (purple blue for nucleus identification), FITC (green) or triple labeling CY5 (red) of hMSCs to determine whether the cells of bone marrow origin express neural specific markers for neurons (NeuN, Tuj-1), astrocytes (GFAP), and oligodendrocytes (MBP). Cellular nuclei were stained by DAPI. In cultures stained for immunoreactivity, 0.2 to 0.5% of the hMSCs expressed NeuN protein, and 6 to 10% of the hMSCs were labeled by the Tuj-1 phenotype. NeuN and Tuj-1 immunoreactivity was colocalized in same cells (pink). 4 to 7% of hMSCs-derived cells expressed GFAP immunoreactivity. 3 to 5% of hMSCs-derived cells expressed MBP immunoreactivity. All of the various concentrations of TBI extracts examined induced hMSCs to express neural phenotype immunoreactivity.

Secretion of Growth Factors by hMSCs Treated with TBI Tissue Extract Supernatant

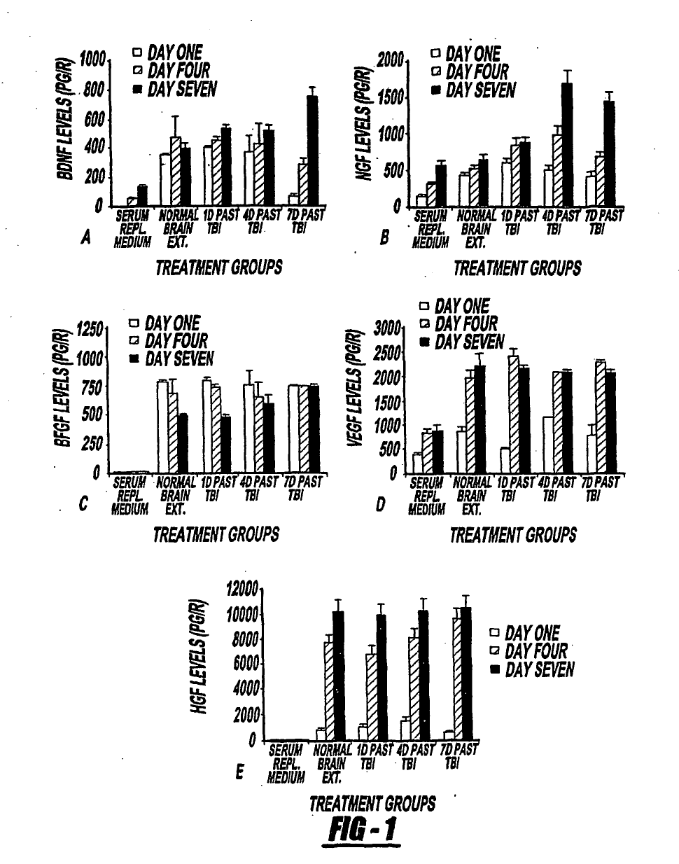

Growth factor secretions by hMSCs after 1, 4 and 7 days in the knockout DMEM with 20% knockout serum replacement medium and containing 20% TBI extract supernatant are shown in FIG. 1. The normal brain and TBI tissue extracts influenced the hMSC secretions of BDNF (FIG. 1a), NGF (FIG. 1b), bFGF (FIG. 1c), VEGF (FIG. 1d) and HGF (FIG. 1e) in vitro. The normal brain tissue extract increased the secretions for all detected growth factors in vitro compared with the medium—alone control. In each experimental group, BDNF, NGF and HGF secretion increased from day 1 through day 7 in conditioned TBI extracts. VEGF secretion was similar for normal brain and post TBI brain groups. VEGF secretion was consistently larger for day 4 and day 7 durations in culture than for day 1 in culture. The profiles for bFGF secretion differed from other trophic factors. Day 1 duration in culture bFGF secretion values, in contrast to other growth factors, exceeded or was equal to secretions for day 4 and day 7 values. These data indicate that TBI promotes the secretion of NGF and BDNF by hMSCs in vitro and that all neurotrophin, and growth factors tested showed a significant increase of hMSC secretion in normal brain compared to hMSCs in serum replacement medium.

Discussion

Human bone marrow stromal cells treated with TBI extracts morphologically can differentiate into neural-like cells and express proteins phenotypic of cerebral parenchymal cells. hMSCs secrete BDNF, NGF, bFGF, VEGF, HGF, and secretion levels depend both on the time of exposure to TBI extracts in culture and the time at which TBI tissue was extracted.

The data demonstrate that hMSCs can be driven to resemble sub-populations of morphologically neural-like cells by exposure to TBI tissue extracts in vitro. Treated hMSCs also express specific cerebral protein markers such as, NeuN (for neurons), Tuj-1 (for early differentiation and neurite outgrowth), GFAP (for astrocytes) and MBP (for oligodendrocytes). Thus, hMSCs is capable of differentiating along multiple cell lineages. Studies have reported that MSCs can be driven to differentiate into neuron-like cells in culture by reagents (Sanchez-Ramos et al., 2000; Woodbury et al., 2000; Deng et al., 2001) and in injured CNS (Azizi et al., 1998; Kopen et al., 1999; Chopp et al., 2000; Li et al., 2000; Chen et al., 2001; Lu et al., 2001b; Lu et al., 2001a; Mahmood et al., 2001). The data show for the first time that some hMSCs when placed in vitro within a specific microenvironment containing TBI tissue extract, respond by assuming morphological as well as phenotypic characteristics of cerebral parenchymal cells. Upon therapeutic transplantation, these cells can provide a source of cellular replacement in the TBI damaged brain.

Bone marrow stromal cells are required for normal hematopoiesis. A number of soluble factors secreted by MSCs that mediate hematopoiesis have been characterized (Berezovskaya et al., 1995; Majumdar et al., 1998; Majumdar et al., 2000). MSCs produce IL-6, -7, -8, -11, -12, -14, -15 and Flt-3 ligand, and induce steady-state levels of M-CSF, G-CSF, GM-CSF and SCF. However, these factors alone are unlikely to provide the mechanism underlying the therapeutic benefit of MSC treatment of TBI. The existence of other, still unknown stromal factors has been postulated. In the experiments presented here, the quantitative ELISA data demonstrate that hMSCs treated with TBI tissue extracts concomitantly secrete BDNF, NGF, bFGF, VEGF and HGF in a manner dependent both on culture time as well as upon the time at which TBI tissue extract was obtained. Intravenous administration of BDNF reduces injury volume after TBI in rats and supports the neuroprotective role for BDNF in brain injury (Koliatsos et al., 1993; Bullock et al., 1999). The neuroprotective potential after NGF injection, or via implantation of NGF-producing fibroblasts and NGF transgenic mice, have been demonstrated in different paradigms of experimental brain injury (Hefti, 1986; Kromer, 1987; Caneva et al., 1995; Gage, 2000). Intravenous administration of bFGF reduced infarct volume in models of focal cerebral ischemia in rats, mice, and cats (Sugimori et al., 2001). VEGF, the strong promoter of angiogenesis, also stimulates axonal outgrowth, nerve cell survival and Schwann cell proliferation (Sondell et al., 1999). The increase in VEGF following crush lesion of the sciatic nerve suggests that VEGF plays a role in nerve regeneration (Sondell and Kanje, 2001). Treatment of experimental stroke in the rat with VEGF significantly reduces functional deficits (Zhang et al., 2000b). hMSCs constitutively produce HGF (Takai et al., 1997), and HGF is an important molecule for tissue repair (Mizuno et al., 2000). Therefore, the findings strongly show that hMSCs are sensitive to the normal brain and the TBI environments and respond by significantly increasing the production of many factors. Given the survival of transplanted MSCs in the traumatically injured neural tissue (Lu et al., 2001b; Lu et al., 2001a; Mahmood et al., 2001), a continuous and microenvironmentally responsive secretion of neuroprotective and angiogenic factors by MSCs at the local-level of compromised tissue is key in the functional benefit provided by MSC transplantation.

The data shows that adult MSCs can be induced to overcome their mesenchymal commitment and constitutes an abundant and accessible brain cellular and molecular reservoir for the treatment of a variety of neurological diseases. The results here show that transplanted MSCs provide functional benefit after TBI (Lu et al., 2001b; Lu et al., 2001a; Mahmood et al., 2001). In particular, MSCs can be readily obtained from a small volume of bone marrow from the patient's own iliac crest and expanded in culture. Therefore, MSCs provide an easily accessible and replenishable source of autologous cells for transplantation. These cells in injured tissue provide a continuous source of vital growth factors for repair and plasticity of injured brain.

FIG. 1 shows the secretions of growth factors of BDNF (FIG. 1A), NGF (FIG. 1B), bFGF (FIG. 1C), VEGF (FIG. 1D) and HGF (FIG. 1E) from hMSCs treated with TBI tissue extract supernatant. The secretions are quantitated with ELISA. The normal brain tissue extract increased the secretions of all detected growth factors in vitro compared with the medium—alone control. In each experimental group, BDNF, NGF and HGF secretion increased from day 1 through day 7 in conditioned TBI extracts. VEGF secretion was similar for normal brain and post TBI brain groups. VEGF secretion was consistently larger for day 4 and day 7 durations in culture than for day 1 in culture. The profiles for bFGF secretion differed from other trophic factors. Day 1 duration in culture bFGF secretion values, in contrast to other growth factors, exceeded or was equal to secretions for day 4 and day 7 values.

### Example 2

Methods:

Rats were subjected to transient middle cerebral artery occlusion and IV injected with 3×106 hMSC 1 day after stroke. Functional outcome was measured before and 1, 7, and 14 days after stroke. Mixed lymphocyte reaction and the development of cytotoxic T lymphocytes measured the immune rejection of hMSC. A monoclonal antibody specific to human cellular nuclei (mAb1281) was used to identify hMSC and to measure neural phenotype. ELISA analyzed neurotrophin levels in cerebral tissue from hMSC-treated or nontreated rats. Bromodeoxyuridine injections were used to identify newly formed cells. Results: Significant recovery of function was found in rats treated with hMSC at 14 days compared with control rats with ischemia. Few (1 to 5%) hMSC expressed proteins phenotypic of brain parenchymal cells. Brain-derived neurotrophic factor and nerve growth factor significantly increased, and apoptotic cells significantly decreased in the ischemic boundary zone; significantly more bromodeoxyuridine-reactive cells were detected in the subventricular zone of the ischemic hemisphere of rats treated with hMSC. hMSC induced proliferation of lymphocytes without the induction of cytotoxic T lymphocytes.

### CONCLUSION

Neurologic benefit resulting from hMSC treatment of stroke in rats can derive from the increase of growth factors in the ischemic tissue, the reduction of apoptosis in the penumbral zone of the lesion, and the proliferation of endogenous cells in the subventricular zone.

Bone marrow stromal cells (MSC; also referred to as mesenchymal stem and progenitor cells) are multipotent and capable of aiding the repair of tissues in vitro and in vivo. MSC normally give rise to bone, cartilage, and mesenchymal cells, and MSC can differentiate into myocytes, hepatocytes, glial cells, and neurons. MSC can pass through the blood-brain barrier and migrate throughout forebrain and cerebellum. Male-derived bone marrow cells systemically infused into female ischemic rats migrate preferentially to the ischemic cortex. Male mouse bone marrow cells administered to irradiated female mice enter the brain over days to weeks and differentiate into microglia and astroglia.

No neuroprotective reagent has improved outcome following stroke. Therapeutic benefit of human MSC (hMSC) for myocardial ischemia and cardiac disease in rats appears to derive from replacement of tissue and the induction of angiogenesis and vasculogenesis. MSC secrete a number of growth factors and cytokines, which normally support hematopoietic progenitors to proliferate and differentiate. Bone marrow contains various primitive cells that secrete several angiogenic growth factors including VEGF and bFGF. Thus, MSC can develop into viable therapy for treating neurologic diseases. There has been demonstrated significant functional recovery in a rat model of middle cerebral artery occlusion (MCAO) when treated with rodent MSC.

Materials and Methods.

hMSC preparation and growth kinetics in vitro. To examine the cell growth kinetics and expansion of hMSC in vitro, bone marrow aspirates were obtained by puncture of the posterior iliac crest of three healthy human donors under local anesthesia. Mononuclear cells of bone marrow specimens (15 to 16 mL per person) were separated on a Ficoll density gradient (Ficoll-Paque [density, 1.073], Pharmacia, Calif.). Isolation and establishment of hMSC cultures were carried out as described by Digirolamo et al. Briefly, mononuclear cells were plated at a concentration of 1×106 cells/75 cm2 tissue culture flasks in 20 mL low-glucose Dulbecco modified Eagle medium (Gibco-BRL, Grand Island, N.Y.) and were supplemented with 20% fetal bovine serum (Gibco-BRL), 100 units/mL penicillin, 100 μg/mL streptomycin, and 2 mmol/L L-glutamine. After 72 hours of incubation, nonadherent cells were removed from the cultures, and fresh culture medium was added to the flasks. The plastic-adherent hMSC were split on day 14 (90% confluence) and every 7 days after that to assess cell growth and cell yield. Nucleated marrow cells were counted using a cytometer to ensure adequate cell number for transplantation. A dose of 3×106 hMSC was injected into each rat. hMSC harvested from five passages and further cultured in the knockout Dulbecco modified Eagle medium (serum free; Gibco-BRL) with 20% knockout serum replacement medium (Gibco-BRL) were used for ELISA-measurement (n=6). The secretion of brain-derived neurotrophic factor (BDNF) and nerve growth factor (NGF) by hMSC was measured at 1, 4, and 7 days in the serum-free Dulbecco modified Eagle medium.

Mixed lymphocyte reaction between rat spleen cells and hMSC in vitro. To study antigen-induced lymphocyte proliferation, 2×105 spleen cells from healthy rats or rats injected with 3×106 hMSC 2 weeks earlier were cultured in triplicate with or without irradiated (20 Gy) hMSC for 96 hours at a 10:1 responder (spleen cells)-to-stimulator (hMSC) ratio. Mixed cells were pulsed with 3H-thymidine (0.25 μCi/well) for 16 hours. The induction of proliferation of splenic lymphocytes by hMSC was measured by the incorporation of 3H-thymidine into replicating splenic cells. Cultures were harvested with an automatic cell harvester, and incorporation of 3H-thymidine was measured by liquid scintillation.

Insert Equation

Rat Cytotoxic T Lymphocyte Response to hMSC In Vitro.

T lymphocytes are implicated as an initiator of graft-versus-host fatal iatrogenic disease. Therefore, human graft-versus-rat host T cell response was measured using a 51Cr assay to determine the lytic effect. Healthy rat spleen cells or spleen cells of rats injected with 3×106 hMSC 2 weeks earlier were cultured with irradiated hMSC for 5 days at a 10:1 responder (spleen cells)-to-stimulator (hMSC) ratio. At the end of the incubation period, viable cells were recovered from the cultures and tested for cytotoxicity to 51Cr-labeled hMSC in an 8-hour 51Cr-release assay.

Animal MCAO Model.

Adult male Wistar rats (weighing 270 to 300 g) were purchased from Charles River Breeding Company (Wilmington, Mass.). Rats were initially anesthetized with 3.5% halothane and maintained with 1.0% to 2.0% halothane in 70% N2O and 30% O2 using a face mask. Rectal temperature was maintained at 37° C. throughout the surgical procedure using a feedback-regulated water heating system. Transient MCAO was induced using a method of intraluminal vascular occlusion modified in the laboratory. The right common carotid artery, external carotid artery, and internal carotid artery were exposed. A length of 4-0 monofilament nylon suture (18.5 to 19.5 mm), determined by the animal weight, with its tip rounded by heating near a flame, was advanced from the external carotid artery into the lumen of the internal carotid artery until it blocked the origin of the MCA. Two hours after MCAO, animals were reanesthetized with halothane, and reperfusion was performed by withdrawal of the suture until the tip cleared the lumen of the external carotid artery.

Experimental Groups.

Group 1.

To measure neurotrophins, rats were subjected to MCAO without treatment (n=3) or were injected with 3×106 MSC (n=3) or 3×106 liver fibroblasts (n=3) in a 1-mL total fluid volume into the tail vein 1 day after stroke. The liver fibroblast study is a restricted “control,” in which fibroblasts were collected from the same strain of Wistar rats to avoid unexpected immune response of control cells to the host rats. Rats were killed 7 days after MCAO for measurement of neurotrophins. Three healthy rats were also used as control subjects.

Group 2.

Rats were subjected to MCAO with 3×106 hMSC (n=9) or 3×106 rat liver fibroblasts (n=9; control) injected at 1 day or MCAO alone without cell donors (n=10; control). Rats were killed 14 days after MCAO for measurement of cellular morphology. Because MCAO induces proliferation of endogenous neural stem and progenitor cells in the ependyma and subependymal zone (also referred to as the ventricular zone/subventricular zone [VZ/SVZ]), 17 rats in Group 2 received daily intraperitoneal injections of bromodeoxyuridine (BrdU, a thymidine analog that labels newly synthesized DNA [50 mg/kg]; Sigma, St. Louis, Mo.) consecutively for 14 days after MCAO with or without IV injection of donor cells for identification of cell proliferation. As a control, an additional two healthy animals were given 14 daily injections of 50 mg/kg BrdU intraperitoneally before death.

Behavioral Testing.

All animals underwent behavioral tests before MCAO and 1, 7, and 14 days after MCAO by an investigator who was blinded to the experimental groups. To measure forelimb somatosensory asymmetries, small adhesive-backed paper dots (113.1 mm2) were used as bilateral tactile stimuli and applied to the radial aspect of the wrist of each forelimb on five trials per day in the home cage. The times at which the rat contacted and removed the stimuli were recorded. Individual trials were separated by at least 5 minutes. The animals were trained in the adhesive-removal dot test for 3 days prior to surgery. Once the rats were able to remove the dots within 10 seconds, they were subjected to MCAO. A modified neurologic severity score (mNSS) was used to grade various aspects on neurologic function. mNSS is a composite of the motor (muscle status and abnormal movement), sensory (visual, tactile, and proprioceptive), and reflex tests.

TABLE 1
Modified neurologic severity score test

| Motor tests | Points |
|---|---|
| Raising the rat by the tail | 3 |
| 1 = Flexion of forelimb | |
| 1 = Flexion of hindlimb | |
| 1 = Head moved >10° to the vertical axis | |
| within 30 s | |
| Walking on the floor (normal = 0; maximum = 3) | 3 |
| 0 = Normal walk | |
| 1 = Inability to walk straight | |
| 2 = Circling toward the paretic side | |
| 3 = Fall down to the paretic side | |
| Sensory tests | 2 |
| 1 = Placing test (visual and tactile test) | |
| 1 = Proprioceptive test (deep sensation, pushing | |
| the paw against the table edge to stimulate | |
| limb muscles) | |
| Beam balance tests (normal = 0; maximum = 6) | 6 |
| 0 = Balances with steady posture | |
| 1 = Grasps side of beam | |
| 2 = Hugs the beam and one limb falls down from | |
| the beam | |
| 3 = Two limbs fall down from the beam or spins | |
| on the beam (>60 s) | |
| 4 = Attempts to balance on the beam but falls | |
| off (>40 s) | |
| 5 = Attempts to balance on the beam but falls | |
| off (>20 s) | |
| 6 = Falls off: no attempt to balance or hang on | |
| to the beam (<20 s) | |
| Reflexes absence and abnormal movements | 4 |
| 1 = Pinna reflex (a head shake when touching the [symbol] | |

Modified Neurologic Severity Score Test
Extract Preparation from the Ischemic Brain:

Seven days after MCAO, rats in Group 1 were anesthetized with halothane; brains were removed, and the ischemic hemispheres were dissected on ice. The samples were then stored at −80° C. Subsequently, each tissue sample was homogenized in 1 g/mL homogenate buffer. The homogenate was centrifuged (10,000 g) for 10 minutes at 4° C., and the supernatant was collected for secretion measurement.

Measurement of Secretion of Growth Factors Using a Sandwich ELISA.

The BDNF ELISA kit was obtained from R & D Systems (Minneapolis, Minn.), and ELISA was prepared as directed by the manufacturer. The ELISA solution was made for NGF. Anti-β(2.5S, 7S) NGF monoclonal antibody, anti-β(2.5S, 7S) NGF-β-gal, and NGF-β standard were purchased from Roche Molecular Biochemicals (Indianapolis, Ind.). In brief, the supernatant collected from the ischemic tissue or the serum-free culture medium from the hMSC was divided into 100- to 200-μL triplicate samples. Monoclonal antibodies to BDNF and NGF were used according to the manufacturer's instructions. Subsequently, the second specific polyclonal antibody to each primary antibody was added. Following an incubation period with a chromogenic substrate, color develops in proportion to the amount of growth factors and is measured using a microplate reader (450 to 620 nm).

Histologic, Immunohistochemical, and Apoptotic Assessment.

Slide Preparation.

Group 2 rats allowed to survive for 14 days after MCAO were used for morphologic analysis. At that time, rats were anesthetized with ketamine (44 to 80 mg/kg intraperitoneally) and xylazine (13 mg/kg intraperitoneally), and the vascular system was transcardially perfused with heparinized phosphate-buffered saline (PBS) followed by 4% paraformaldehyde in PBS. The brains were immersed in 4% paraformaldehyde in PBS for 2 days, and then the brain tissues were cut into seven equally spaced (2 mm) coronal blocks. The tissues were processed, and 100-μm-thick free-floating vibratome coronal slides from each block (five vibratome slides per block) were cut. All remaining brain blocks were embedded in paraffin, and a series of adjacent 6-μm-thick slides were cut.

Measurement of Infarct Volume.

One of each coronal paraffin slides (6 μm thick) from seven blocks was stained with hematoxylin-eosin (H-E). The seven brain slides were traced using the Global Lab Image analysis system (Data Translation, Malboro, Mass.). The indirect lesion area, in which the intact area of the ipsilateral hemisphere was subtracted from the area of the contralateral hemisphere, was calculated. The lesion volume is presented as a volume percentage of the lesion compared with the contralateral hemisphere.

Immunohistochemical Staining.

After blocking in normal serum, all vibratome slides were treated with the monoclonal antibody specific to human nuclei (mAb1281; Chemicon, Temecula, Calif.) diluted at 1:100 in PBS for 3 days at 4° C. Following sequential incubation with fluorescein isothiocyanate-conjugated rabbit antibody to mouse IgG (dilution, 1:100; Dakopatts, Calif.), the secondary antibody was bound to the first antibody to mAb1281. Cells derived from hMSC were identified using morphologic criteria and immunohistochemical staining with mAb1281 present in the donor cells but not present in the parenchymal cells. To visualize the cellular colocalization of mAb1281 and cell-type-specific markers in the same cells, double staining was used on serial reference vibratome slides (100 μm) centered at the ischemic core (coordinates at bregma −1.0 1.0 mm). Each coronal slide was treated with the first primary antibody, mAb1281, as described above and then was treated with cell-type-specific secondary primary antibodies conjugated to cyanine-5.18 (Calbiochem, Calif.) for 3 days at 4° C.: a neuronal nuclear antigen (NeuN for neuronal nuclei [dilution, 1:200]; Chemicon), microtubule-associated protein 2 (MAP-2 for neuronal dendrites [dilution, 1:200]; Sigma), glial fibrillary acidic protein (GFAP for astrocytes [dilution, 1:1,000]; DAKO, Carpinteria, Calif.), and vWF (for endothelial cells [dilution, 1:400]; DAKO). Negative control slides for each animal received identical preparations for immunohistochemical staining, except that primary antibodies were omitted.

Laser-Scanning Confocal Microscopy.

Coronal vibratome slides were analyzed with a Bio-Rad MRC 1024 (argon and krypton) laser-scanning confocal imaging system mounted onto a Zeiss microscope (Bio-Rad, Cambridge, Mass.). For immunofluorescence-labeled slides, green (fluorescein isothiocyanate) and red (cyanine-5.18) fluorochromes on the slides were excited by the laser beam at 488 nm and 647 nm, and emissions were acquired sequentially with a photomultiplier tube through 522-nm and 670-nm emission filters. The total number of mAb1281-positive cells was measured on five sequential slides (100 μm thick) for each block from all seven blocks by using XYZ stage encoders for cell counting.26 The total number of mAb1281-positive cells of the whole forebrain was then calculated by summing numbers of mAb1281-positive cells from all seven blocks. A total of 500 mAb1281-positive cells per animal were counted to obtain the percentage of mAb1281-positive cells colocalized with cell-type-specific markers (NeuN, MAP-2, vWF, and GFAP) by double staining.

Apoptotic Cell Staining.

Five coronal paraffin slides (6 μm thick; 25-μm interval) from the above-referenced block coordinated at bregma −1.0 1.0 mm were used for apoptotic cell analysis. These slides were stained by the terminal deoxynucleotidyl transferase-mediated dUTP-biotin nick end-labeling (TUNEL) method for in situ apoptosis detection (ApopTag kit; Oncor, Gaithersburg, Md.). After quenching endogenous peroxidase activity with H₂O₂ in PBS, slides were placed in terminal deoxynucleotidyl transferase. Anti-digoxigenin-peroxidase was applied to the slides, and peroxidase was detected with 3,3′-diaminobenzidine. After TUNEL staining, the slides were counterstained with Mayer hematoxylin. Negative control slides were run from every block. In TUNEL preparations, only cells containing dark brown apoptotic bodies (>2) were referred to as apoptotic cells.

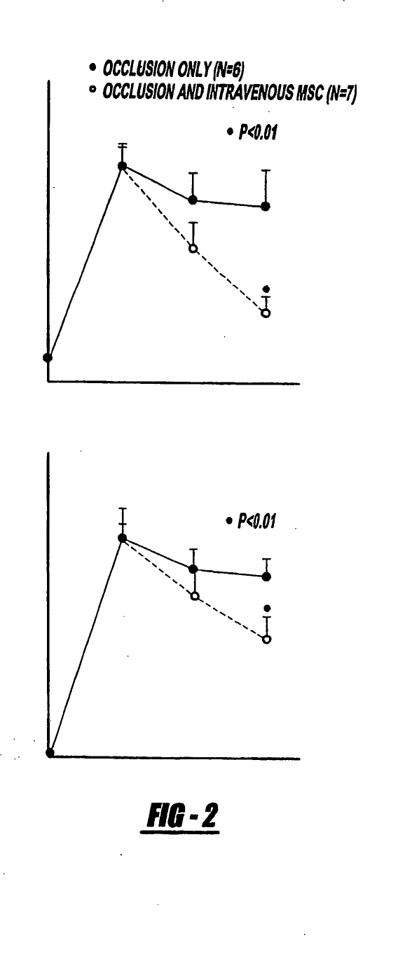

FIG. 2 depicts a standard coronal section identified at the level of the anterior commissure of rat brain, which divides the right hemisphere into three subregions (ischemic core, ischemic boundary zone, and VZ/SVZ). Exogenous hMSC (mAb1281) was measured, cell-type-positive cells (NeuN, MAP-2, GFAP, and vWF), and apoptotic cells (TUNEL-positive cells) in these regions of the ipsilateral and contralateral hemispheres. Histologic features with routine H-E staining were used to identify three regions: the ischemic core (diffuse pallor of the eosinophilic background) and the inner (vacuolation or sponginess of the neuropil) and the outer boundary zones (from sponginess to entirely intact tissue [most cells were intact; however, scattered injured and dead cells could be observed]) of the ischemic lesion, and alterations in the shape and stain ability of cells. FIG. 2 shows a standard coronal section identified at the level of the anterior commissure of rat brain that divides the right hemisphere into three subregions (ischemic core [IC]; ischemic boundary zone [IBZ]; and ventricular zone/subventricular zone [VZ/SVZ]) and eight fields (1, the cortex in IC; 2, the striatum in IC; 3-4, the cortex in IBZ; 5-6, the striatum in IBZ; and 7-8, the striatum in VZ/SVZ) for analysis of response to treatment.

Statistical Analysis.

All measurements were performed blindly. The behavior scores (from the adhesive-removal dot test and the mNSS) were evaluated for normality. Repeated measure analysis was conducted to test the treatment effect on the behavior score. The analysis began with testing for the treatment-time interaction at the significance level of 0.1; testing for the overall treatment effect was done if there was no interaction detected at the significance level of 0.05. A subgroup analysis of the treatment effect on each behavior score at each time was conducted at the significance level 0.05, if a treatment-time interaction at the significance level of 0.1 or an overall treatment effect at the significance level of 0.05 was present. Otherwise, subgroup analyses were considered as exploratory. Student's t-tests were used to evaluate differences between the control group and the treated group in terms of the lesion volume and cell numbers. The ELISA data were linearized by plotting the log of BDNF and NGF concentrations vs the log of the optical density, and the best-fit line was determined by regression analysis. Average duplicate readings were made for each standard, control, and sample, and the average zero standard optical density was subtracted. The means (SD) and p value for testing the difference between treated and control groups are presented.

Results.

Growth Kinetics of hMSC In Vitro.

Bone marrow-derived hMSC from three healthy human donors were tested by culture expansion. In the primary cultures, hMSC grew as a morphologically homogeneous population of fibroblast-like cells. During subsequent passages, usually at 7-day intervals, hMSC grew as whorls of densely packed spindle-shaped cells. At the end of 5 weeks (four passages), the hMSC yield ranged between 5.4 and 6.6×107 cells (table 2).

TABLE 2
Growth kinetics of hMSC

| Donor | Bone marrow, | Mononuclear | hMSC (10⁶) (7 d per each passage) | | | |
|---|---|---|---|---|---|---|
| no. | mL | cells, 10⁶ | Passage 1 | Passage 2 | Passage 3 | Passage 4 |
| 1 | 16 | 100 | 1.65 | 9.0 | 18.9 | 53. [symbol] |
| 2 | 16 | 130 | 3.21 | 14.3 | 35.8 | 64. [symbol] |
| 3 | 15 | 160 | 10.8 | 18.9 | 28.8 | 66. [symbol] |

hMSC = human bone marrow stromal cells.

Mixed lymphocyte reaction and cytotoxic T lymphocyte response between rat spleen cells and hMSC in vitro.hMSC significantly increased the proliferation of healthy rat spleen cells (stimulation index=18.8) compared with unstimulated spleen cells (FIG. 2A). The proliferation of spleen cells from rats injected with hMSC was also increased following restimulation with hMSC in vitro (stimulation index=15.6); however, the proliferative response in these cells was not significantly different from that in the spleen cells of a healthy rat. These data indicate that although hMSC are capable of inducing a primary proliferative response in rat spleen lymphocytes, administration of hMSC to rats fails to sensitize lymphocytes in vivo for a secondary in vitro proliferative response.

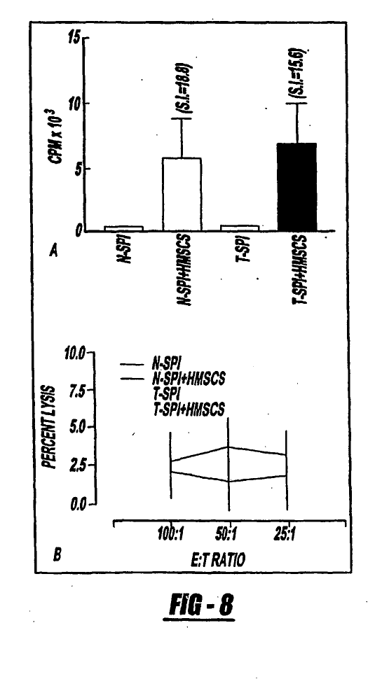

FIG. 8A shows mixed lymphocyte reaction between rat spleen cells and human bone marrow stromal cells (hMSC): 2×105 healthy rat spleen cells (N-Spl) or spleen cells from rats treated IV with hMSC (T-Spl) 2 weeks earlier were cultured in triplicate with or without irradiated (20 Gy) hMSC for 96 hours at a 10:1 responder:stimulator ratio. Cultures were pulsed with 3H-thymidine (0.25 μCi/well) for 16 hours and then harvested with an automatic cell harvester. The incorporation of 3H-thymidine was measured by liquid scintillation. No differences were detected between spleen cells obtained from hMSC-treated and nontreated rats. SI=stimulation index. (B) Rat spleen cells (1×107) were cultured with 1×106 irradiated (20 Gy) hMSC for 5 days. At the end of the incubation period, viable cells were recovered from the cultures and tested for cytotoxicity to 51 Cr-labeled hMSC in an 8-hour 51Cr-release assay at effector:target (E:T) ratios. Rat spleen cells did not generate a cytotoxic T lymphocyte response to hMSC. All values are expressed as means±SD. FIG. 8B demonstrates <4% lysis of target cells (hMSC) by healthy rat spleen cells incubated with or without the stimulators (hMSC). Similarly, the priming of spleen cells in vivo by administration of hMSC followed by restimulation with hMSC in culture for 5 days failed to evoke cytotoxicity in them, indicating that hMSC fail to induce a cytotoxic T lymphocyte response in rat spleen cells.

Neurologic Functional Testing.

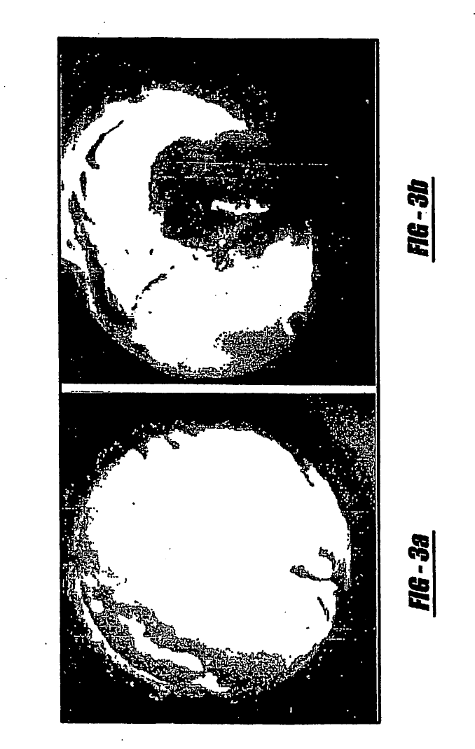

At 14 days after stroke, functional recovery shown by the adhesive-removal dot test (p<0.05; FIG. 3A) and the mNSS test (p<0.05; see FIG. 3B) was found in rats injected with 3×106 hMSC 1 day after MCAO compared with control rats subjected to MCAO alone and rats injected with 3×106 rat liver fibroblasts.

FIG. 3 shows the results of behavioral functional tests (A: adhesive-removal dot test; B: modified neurologic severity score [mNSS] test) before and after middle cerebral artery occlusion (MCAO). Rats were subjected to 2 hours of MCAO alone (n=10) or were injected with cultured human bone marrow stromal cells (hMSC) (n=9) or rat liver fibroblast cells (LC; n=9) 1 day after MCAO. Significant functional recovery was detected in rats treated with hMSC compared with control subjects. Open circle=MCAO; filled circle=+LC; triangle=+hMSC.

Sandwich ELISA Quantitation.

Using sandwich ELISA methods, the secretion levels of BDNF (969±198 pg/mL vs 434±59 pg/mL and 498±76 pg/mL) and NGF (1,227±111 pg/mL vs 834±123 pg/mL and 980±55 pg/mL) were increased (p<0.05) in the ischemic hemisphere of hMSC-treated rats compared with animals 7 days after MCAO alone without cell treatment and rats treated with rat liver fibroblasts. In vitro data indicate that hMSC secrete BDNF and NGF in a time-dependent manner. A significant increase in BDNF and NGF was detected in the serum-free medium at 4 and 7 days in culture compared with 1 day (table 3).

TABLE 3
Neurotrophin secretion by hMSC in culture

| | Mean protein level ± SD, |
|---|---|
| Neurotrophin time, d | pg/mL |
| BDNF | |
| 1 | 0 ± 0 |
| 4 | 57 ± 12* |
| 7 | 141 ± 28* |
| NGF | |
| 1 | 162 ± 22 |
| 4 | 321 ± 74* |
| 7 | 581 ± 147* |

An increase in BDNF and NGF was detected in the serum-free medium at 4 and 7 days in culture compared with 1 day in culture.
*p < 0.05.
hMSC = human bone marrow stromal cells;
BDNF = brain-derived neurotrophic factor;
NGF = nerve growth factor.

Morphologic Analysis.

Rats subjected to 2 hours of MCAO were infused with 3×106 hMSC 1 day after ischemia and killed 14 days after MCAO for morphologic analysis. Within the coronal slides stained with H-E, dark and red neurons were observed in the ischemic core of all rats subjected to MCAO with and without hMSC injection. No significant reduction in the volume of ischemic damage was detected in hMSC-treated rats (lesion volume, 33.3%±7.6%) compared with control rats subjected to MCAO alone (36.3%±10.5%) or rats injected with rat liver fibroblasts 14 days after MCAO (34.6%±9.1%).

Within the brain tissue, cells derived from hMSC were characterized by round-to-oval nuclei identified by the human specific antibody mAb1281. hMSC (124×103±46×103; 4% of 3×106 hMSC) survived and were distributed throughout the ischemic damaged brain of recipient rats. Although mAb1281-reactive cells were observed in multiple areas of the ipsilateral hemisphere, including the cortex and striatum, most mAb1281-labeled hMSC (60% of the total of 124×103±46×103) were located in the ischemic boundary zone. A few cells were also observed in the contralateral hemisphere (9×103±2×103; 0.3% of 3×106 hMSC).

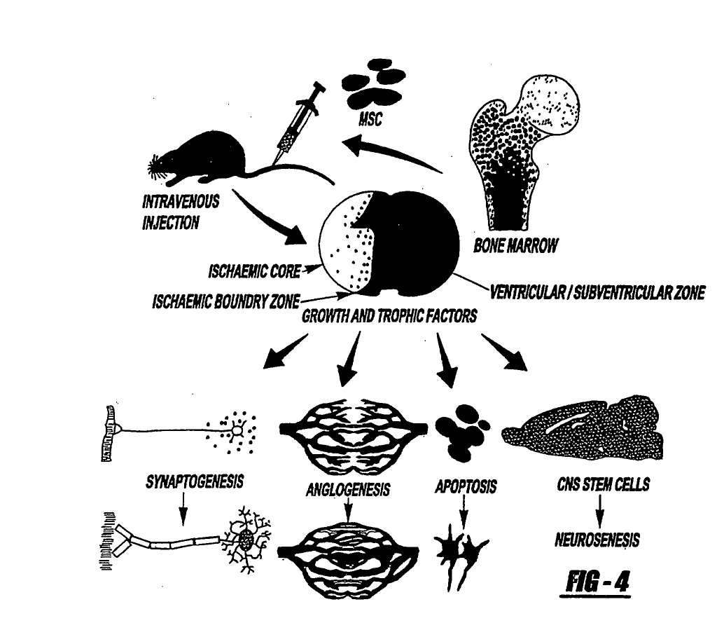

Double staining immunohistochemistry revealed that few mAb1281-positive cells were reactive for the neural markers used. Percentages of mAb1281-labeled hMSC that expressed NeuN, MAP-2, GFAP, and vWF were 1%, 1%, 5%, and 2%. Laser scanning confocal microscopy images showed colocalization of the monoclonal antibody specific to human nuclei mAb1281 (green for hMSC identification) with NeuN, MAP-2, GFAP, or vWF (red for cell-type-specific markers) in the recipient rat brain (FIG. 4, a through h). Most mAb1281-positive cells encircle vessels, with few cells located in the parenchyma.

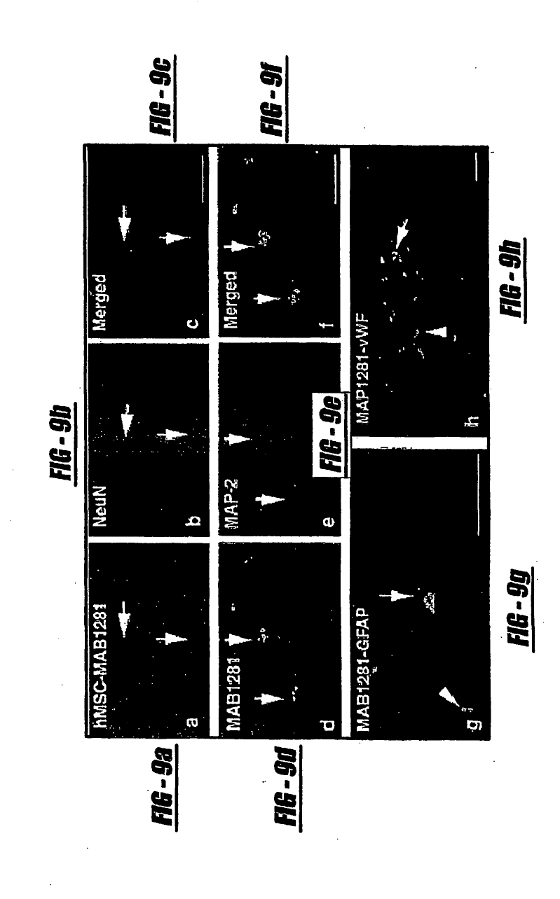

FIG. 9 shows photomicrographs showing the morphologic characteristics of exogenous human bone marrow stromal cells (hMSC) and endogenous brain cells in rat brain. Using double immunofluorescent staining, mAb1281 (the monoclonal antibody specific to human nuclei)-reactive cells were present in the damaged region of the brain. Laser scanning confocal microscopy images showed mAb1281 (green for hMSC [a,c,d,f-h]), neuronal nuclear antigen (NeuN) (b,c), microtubule-associated protein 2 (MAP-2) (e,f), glial fibrillary acidic protein (GFAP) (g), and vWF (h) (red for cell-type-specific markers) in the recipient rat brain. Scale bar=50 μm.

Using TUNEL (FIG. 7, a, c, and d) and H-E staining (see FIG. 7b), apoptotic cells with typical dark brown rounded or oval apoptotic bodies were counted in the ischemic boundary zone. Within the reference coronal 6-μm-thick section, the number of apoptotic cells measured was reduced (38.5±3.4 vs 82.6±3.8 or 76.4±6.8; p<0.05) in the ischemic boundary zone in hMSC-treated rats compared with animals 14 days after MCAO alone or ischemic rats treated with liver fibroblasts.

FIG. 7 shows apoptotic cells (a: terminal deoxynucleotidyl transferase-mediated dUTP-biotin nick end-labeling [TUNEL]-positive cells [arrows]; b: hematoxylin-eosin [H&E] staining) are present in an ischemic boundary zone after middle cerebral artery occlusion (MCAO) alone. Decreased apoptotic cells (d: more survival of blue-hematoxylin-counterstained cells; arrowheads) were detected in rats injected with human bone marrow stromal cells (hMSC) compared with rats injected with liver fibroblasts (c). Few bromodeoxyuridine (BrdU; a marker for newly synthesized DNA)-positive cells (arrows) were present in the ventricular zone/subventricular zone (VZ/SVZ) of healthy brain (e). Increased BrdU-positive cells were detected in the VZ/SVZ of the ipsilateral hemisphere of rats subjected to MCAO alone (f) and rats injected with liver fibroblasts (g). Significantly increased BrdU-positive cells were detected in the VZ/SVZ in rats treated with hMSC (h) compared with rats subjected to MCAO with or without liver cell treatment. Scale bar=15 μm.

Few BrdU-positive cells were present in the VZ/SVZ (see FIG. 7, e through h). Significantly more BrdU-reactive cells were detected in the VZ/SVZ of the ipsilateral hemisphere of rats subjected to MCAO with hMSC treatment (see FIG. 7h) than in that of rats subjected to MCAO alone (see FIG. 7f) or rats treated with liver fibroblasts (see FIG. 7g). Five coronal paraffin slides (6 μm thick; 25-μm interval) from the standard reference section with coordinates at bregma −1.0 1.0 mm were used for BrdU-reactive cell analysis. The number of BrdU-positive cells per slide in the VZ/SVZ of rats subjected to MCAO with hMSC treatment (95.3±24.1) was significantly higher than that in the VZ/SVZ of rats subjected to MCAO alone (27.5±18.5) or ischemic rats treated with liver fibroblasts (37.8±11.2). A higher number of BrdU-positive cells per slide expressed NeuN (2.5±0.4 vs 0.5±0.6 or 0.6±0.4; p<0.05) and GFAP (4.4±2.3 vs 1.4±1.1 or 1.7±0.5; p<0.05) for rats subjected to MCAO with hMSC treatment than for rats subjected to MCAO alone or rats treated with liver fibroblasts 14 days after stroke.

Discussion.

IV injection of hMSC 1 day after stroke significantly improved functional outcome according to the somatosensory score and the mNSS compared with rats subjected to MCAO alone or injected with rat liver fibroblasts. This benefit can reflect production of growth factors, including neurotrophins that can promote repair of damaged parenchymal cells, reduce apoptosis in the ischemic boundary zone, and enhance proliferation and differentiation of endogenous neural stem and progenitor cells in the VZ/SVZ after stroke in rats.

Neural grafts have reversed functional deficits associated with brain damage. The present human graft-versus-rat host data are consistent with findings from other studies showing preferential homing of IV transplanted allogeneic bone marrow cells to the site of injury after onset of permanent MCAO in irradiated animals7 and transient 2 hours of MCAO in nonirradiated animals. Morphologic analysis indicates that hMSC have the capacity to selectively migrate into the ischemic damaged rat brain. hMSC survive, and a scattered few express protein markers for parenchymal brain cells.

Though hMSC can have the potential to replace lost neurons, it is likely that the mechanisms providing therapeutic benefit are multipronged. The data show that injection of 3×106 hMSC 1 day after stroke improves functional outcome according to the somatosensory score and the mNSS compared with nontreated rats 7 and 14 days (p<0.01) after administration. However, only 1%, 5%, and 2% of hMSC express neuronal, astrocytic, and endothelial cell proteins, being too soon for full cellular differentiation and integration into tissue. Therefore, a more likely mediator of short-term benefit is that hMSC supplement compromised tissues with array of growth factors that promote functional recovery of the remaining neurons and reduce apoptosis in the ischemic boundary zone. MSC can be directly involved in promoting plasticity of the ischemic damaged neurons or in stimulating glial cells to secrete neurotrophins (e.g., BDNF and NGF). The interaction of hMSC with the host brain can lead hMSC and parenchymal cells to produce abundant trophic factors, which can contribute to recovery of function lost as a result of a lesion.30,31 Using sandwich ELISA methods in this study, there is demonstrated that the secretion levels of BDNF and NGF were significantly increased in the ischemic hemisphere of hMSC-treated rats compared with animals 7 days after MCAO alone without cell treatment and with rat liver cell treatment. Although the presence of BDNF and NGF in the ischemic brain were measured, the possibility that other growth factors (such as angiogenic factors VEGF32 and HGF33) can improve functional recovery at least in part by increasing angiogenesis was not excluded. Angiogenesis is associated with improved neurologic recovery from stroke.

MSC behave as small molecular “factories.” These cells produce an array of cytokines and trophic factors. They also secrete these factors over an extended period and not in a single bolus dose. MSC express many cytokines known to play a role in hematopoiesis and also supply autocrine, paracrine, and juxtacrine factors that influence the cells of the marrow microenvironment itself. It is likely that MSC within cerebral tissue express these factors, and it is the effect of these cytokines and trophic factors on brain tissue, which rapidly and effectively promote restoration of function. These cells when cultured under different ionic microenvironments (e.g., calcium) respond to the cues of the ionic microenvironment by adjusting growth factor expression. This suggests that cells within injured tissue express trophic and growth factors titrated to the needs of the tissue. In the brain, treatment of stroke with MSC produces a variety of trophic factors and cytokines in an anatomically distributed, tissue-sensitive, and temporally ongoing way, in sharp contrast to a single localized injection of a specific factor.

Neural stem cells reside within the VZ/SVZ, and these cells migrate to their destiny in the developing brain. In the healthy adult brain, the absence of forebrain neuronal production can reflect not a lack of appropriate neuronal precursor cells but rather a tonic inhibition and/or a lack of postmitotic trophic and migratory support. In this study, BrdU-reactive cells increased in the VZ/SVZ after MCAO with hMSC treatment compared with MCAO alone, suggesting that IV injected hMSC can stimulate the endogenous brain cells to proliferate and participate in the repair of ischemic damaged brains. These findings are consistent with data obtained using IV administration of MSC derived from the rat.

IV transplantation of hMSC in rats does not sensitize rats against hMSC, as determined by mixed lymphocyte reaction in vitro. Similarly, the spleen cells of healthy rats or rats injected with hMSC fail to generate a cytotoxic T cell response to hMSC, a functional immune response that is implicated in the rejection of foreign organ/cell transplants. These data suggest that immunologic rejection of hMSC by rats is not a concern for testing hMSC as a treatment for stroke. Potentially, and more important, is that the rat spleen demonstrated little or no sensitivity to the injected hMSC. The inability of hMSC to induce a strong immune response can be related to the weak immunogenicity of these cells due to the absence or low expression of major histocompatibility complex (class I and class II) and costimulatory (CD40, CD80, and CD86) molecules. In addition, hMSC can also secrete soluble mediators that downregulate the development immune responses involved in the rejection of a xenograft. These data call for additional studies to investigate the immunogenicity of allogeneic cell-adherent populations of MSC.

The data indicate that IV administered hMSC promote neurologic functional recovery 2 weeks after stroke. hMSC selectively enter in the cerebral ischemic region. The interaction between hMSC and the ischemic brain enhances the secretion of neurotrophins, which can reduce neuronal apoptosis in the ischemic boundary zone and promote cell proliferation from the relatively intact SVZ in the ischemic brain. However, whether the cells originating in the SVZ migrate and integrate into the ischemic brain has not been determined. In the CNS, effective treatment of neural injury can require activation of endogenous compensatory mechanisms including remodeling of cerebral circuits, with the exact mechanisms being uncertain. With elucidation of the mechanisms underlying the MSC-evoked reduction of neurologic deficits as well as demonstration of long-term therapeutic benefit, hMSC can provide a powerful molecular and cellular therapy for stroke and possibly a broad array of human neurologic disorders.

### Example 3

Treatment of Neural Injury: Preclinical Protocols

In investigating the hypothesis that MSC promote functional recovery after stroke, Applicants were confronted with various options for implementing preclinical cellular therapy protocols. Among issues to address were when and where to implant the cells. Since the interest is in restorative therapy, with the hypothesis that the size of the ischaemic lesion is not altered by effective restorative therapy, Applicants initially chose to treat animals 1 day or more after stroke.^{31,32} This timing is clinically reasonable. If deficits persist for a day after a stroke, the event is classified as a stroke and not as a transient ischaemic attack. At 1 day, patients tend to be stabilised, and the severity of the neurological deficits can be easily assessed.

The most direct route of placement of cells into brain is via surgical transplantation. Should the cells be placed within the lesion, in healthy non-ischaemic tissue, or within the boundary zone? Drawing on the observations of the brain, particularly with the boundary zone of a lesion being in a developmental state, in the initial studies Applicants opted to place naïve whole bone-marrow cells within the boundary tissue.³² Thus, cells were extracted from donor rats and surgically and stereotactically implanted into the boundary zone of the ischaemic lesion within subcortical and cortical tissue.³¹⁻³³ The main hypothesis to be tested was that these cells promote functional recovery, so neurological and functional tests were carried out on the animals. A complete neurological examination (table 1) was done. This examination, the modified neurological severity score (mNSS), provides an index of motor, sensory reflex, and muscle status.³⁴⁻³⁸ In addition, Applicants used a somatosensory test, which involves removal of a sticky tab from the paw,³⁹ and a rotarod test,⁴⁰ which measures the time the rat persists on an accelerating treadmill. Measurements were done before stroke and 7 days and 14 days afterwards.³¹ Animals were killed at 14 days, and transplanted cells were sought in the cerebral tissues by histology. One question addressed with this histological analysis was whether MSC differentiate into brain parenchymal cells. Similar experiments were done on mice subjected to embolic occlusion of the middle cerebral artery and treated with intracerebral transplantation of naïve whole bone-marrow cells from donor mice. Functional measurements were made 28 days after transplantation. There was emarkable and rapid functional recovery after placement of these cells within ne boundary of the ischaemic lesion. A similar study of intraparenchymal transplantation of MSC into the striatum of mice in which a Parkinson-like lesion was induced with 1-methyl-4-phenyl-1,2,3,6-tetrahydropyridine showed significant recovery of motor function.⁴² Likewise, MSC were implanted adjacent to a contusion lesion of the spinal cord, and significant functional benefit was evident.

Variations on these experiments showed that coadministration of MSC with trophic factors, such as brain-derived nerve growth factor, promotes functional recovery, and preculture of these cells in growth factors facilitates functional benefit as well as increasing the numbers of cells that express brain-cell phenotypic proteins. Acclimatisation of cells in culture to the environment of the brain seems to ease the transition from in vitro to in vivo. Many of the transplanted bone-marrow cells underwent apoptosis in the ischaemic brain. Therefore, Applicants coadministered with the bone-marrow cells Z-Val-Ala-DL-Asp-fluoromethylketone (Z-VAD), a caspase inhibitor. The hypothesis was confirmed; numbers of apoptotic cells were significantly decreased and function as measured on a rotarod test showed incremental benefit. Thus, even with cellular therapy, adjunctive therapy can improve the desired outcome.

Similar therapeutic interventions were also effective in animal models of traumatic brain injury, spinal-cord injury, and Parkinson's disease; in all three models there was a significant reduction in neurological deficits with the surgical implantation of MSC. Therapeutic benefit became evident within days of transplantation. However, only 1-3% of cells expressed proteins phenotypic of parenchymal cells. Although the proportions of cells expressing such proteins could be increased with preculturing, the numbers of cells transplanted are tiny compared with the amount of hemispheric brain tissue infarcted after occlusion of the middle cerebral artery (roughly 40%). 14 days after occlusion, about 50 000 cells (SE 18 000) or 12.5% of the 400 000 transplanted, survive; a small percentage express neural proteins—far too few to replace the infarcted tissue.

The success of the direct implantation of these cells into brain prompted experiments to test a less invasive vascular route of administration. Rats were subjected to occlusion of the middle cerebral artery, and the carotid artery ipsilateral to the hemisphere with the ischaemic lesion was cannulated for injection of cells. About 2 million MSC were injected 1 day after stroke. A battery of neurological tests was done before and after treatment. Histological analysis showed a paucity of cells expressing proteins phenotypic of parenchymal cells. However, significant functional benefit was evident. Applicants also tested the potential of an arterial route of MSC administration for the treatment of traumatic brain injury. Although cells entered the brain when administered via the carotid route, there was no functional benefit, probably because the route of administration required ligation of the internal carotid artery, causing an imposed hypoperfusion that exacerbated the traumatic brain injury.

Applicants then investigated the feasibility of the more clinically relevant intravenous route of administration. This approach is clearly less invasive and has fewer adverse effects than carotid or direct tissue injection. A venous route also allows for multiple and long-term cell treatments.

Others have shown that cells injected intravenously find their way into the brain. However, there had been no studies showing that in injury, such as stroke or trauma, intravenously injected cells would selectively migrate to the site of ischaemic injury and promote functional benefit. Applicants therefore tested this hypothesis in rats subjected to occlusion of the middle cerebral artery. A day or more after stroke, 1-3 million MSC were injected into a tail vein. Applicants carried out a battery of neurological outcome measures (table 1). Animals in which the cells were administered 1 day after stroke were killed 14 days after stroke (FIG. 1) and those treated 7-days after stroke were killed at 35 days. As in previous experiments, cells were labelled with bromodeoxyuridine, a marker of newly synthesised DNA, to indicate generation of new cells. Also, MSC from male rats were injected into female animals, and the cells identified by in-situ hybridisation to the Y chromosome. The treated animals showed significant functional improvement with treatment (FIG. 2). Control populations of cells were also used to test for the specificity of the cell type in promoting improved function. Dead MSC and liver and lung fibroblasts (as non-mesenchymal cell controls) showed no therapeutic benefit and were no better than a phosphate-buffered saline control. Thus, the intravenous route provides significant functional improvement after stroke and trauma. This was also true for treatment initiated 7 days after stroke, and the functional benefit was similar in male and female rats.

In an effort to resemble the human test conditions more closely, human marrow stromal cells were used as the donor cell population, rather than rat MSC. Human cells were extracted by puncture of the posterior iliac crest of healthy donors under local anaesthesia. Mononuclear cells of the bone-marrow extracts (15-16 mL) were separated. A dose of 3 million human MSC was injected intravenously into each rat, 1 day after occlusion or after traumatic brain injury. Strong functional improvement was found after both stroke and trauma. The human cells are easily obtained from donors. They can be readily expanded to very high numbers, and antibodies are available for separation by flow cytometry or magnetic cell sorting. Human MSC have been used to treat patients with cancer and multiple sclerosis. Thus, safety data in human beings are available.

Applicants did not observe any indication of immunorejection (unpublished observation). The spleens of untreated rats and animals treated with human MSC were removed and cultured with human MSC. The proliferation of spleen cells from rats injected with human MSC increased after restimulation with these cells in vitro; however, the proliferative response did not differ significantly from that in spleen cells from untreated rats. Thus, although human MSC can induce a primary proliferative response in rat splenic lymphocytes, the administration of these cells to rats does not sensitise lymphocytes in vivo for a secondary proliferative response in vitro. T lymphocytes are implicated as an initiator of graft-versus-host disease. Therefore, the response of rat host T cells to human graft was measured with a standard chromium-51 assay to assess the lytic effect. Human MSC did not induce a cytotoxic-T-lymphocyte response in the rat spleen cells. Applicants cannot exclude the possibility that rodents and human beings can respond differently to MSC treatment. However, another possibility is that a universal cell, allogeneic cells, and not autologous cells can be used to treat patients. Clearly, more data in human beings are required to test this hypothesis. Initial clinical application will entail autologous transplantation.

There are still many issues to address, including how these cells are targeted to sites of injury, and how they provide benefit. How do the cells know where to go? What mechanisms target these cells specifically to sites of injury? The most interesting issue, however, is the effects of the cells on the brain and how these effects translate into therapeutic benefit.

Targeting of MSC to Sites of Cerebral Injury

Where do the intravenously injected cells go? First, the injected cells have to be marked so that they can be identified in tissue. MSC can be identified by means of antibody reactivity to various labels. MSC can be labelled with bromodeoxyuridine; male-derived cells can be injected into female animals and the Y chromosome identified by in-situ hybridisation; or human cells can be injected into rats and an antibody to human antigens used. Intravenously injected cells have been found within liver, kidney, spleen, and bone marrow. However, most identified MSC encircle microvessels in these organs, with few cells located in the parenchyma. Very few cells (1.5-3.0% of 3 million injected MSC at 14-35 days after treatment) were detected within the parenchyma of brain tissue. In injured brain, whether after stroke or traumatic injury, the vast majority of cells were targeted to the region of the injury. For example, after stroke, more than 80% of cells were within the affected hemisphere, with the majority of these cells congregating in the areas around the lesion. Many cells were also present adjacent to or within vessels. How do cells target injured tissue, and is the localisation of these cells to microvasculature important?

The homing in of MSC to sites of injury is reminiscent of the response of inflammatory cells to injured tissue. Neutrophils and monocytes target injured and inflamed tissue by an orchestrated sequence of vascular and cellular molecular signalling. Adhesion molecules and their receptors, expressed on the inflammatory cells and the vasculature, guide the cells to injured tissue and transport these cells across the vascular boundary, commonly passing through the blood-brain barrier. These targeting and adhesion molecules work in concert with chemokines. Applicants therefore tested whether adhesion molecules and chemoattractive agents operate and target MSC to brain. Applicants used a Boyden chamber, an assay for cell migration between two chambers separated by a permeable membrane. MSC were adjusted to 5×10 cells/mL in migration medium (Iscove's modified Dulbecco's medium with 5% bovine serum albumin). 50 •L cell suspension was added to each upper well. The number of MSC that migrated to the bottom surface was counted in five optical fields (0·12 mm² area). Since ischaemic brain tissue expresses chemotactic proteins, such as monocyte chemoattractant protein 1 and macrophage inflammatory protein 1, Applicants placed these substances in the lower chamber, to provide a dose-dependent increase in migration. Similar responses were found when adhesion molecules such as intercellular adhesion molecule 1 were placed in the lower chamber. The increased migration was effectively blocked by addition of antibodies to the adhesion molecules or the chemokines to the lower chamber. When tissue from brain subjected to traumatic injury or stroke was placed in the lower chambers, cell migration was also significantly increased. These findings provide an insight into how the cells assume an inflammatory-cell-like identity, and how they “know” to target injured tissue specifically. Thus, any injury that has an inflammatory response, including neurodegenerative processes such as Parkinson's disease and multiple sclerosis, can guide MSC to the affected sites. The dependence of guidance on the degree of injury also provides a form of titration of “effective” dose of cells. The more severe the injury and concomitant inflammatory response, the higher the numbers of cells directed to the site.

Mechanisms of Action

How do the cells affect the brain and thereby promote functional recovery from injury and pathological processes? The possibility that MSC benefit cerebral tissue by becoming brain cells is very unlikely. With intravenous injection and the numbers of intraparenchymal cells numbering a few hundred thousand at most, there are very few cells present, even if they become brain cells, to replace a volume of tissue of more than a few cubic millimetres. Benefit is detected in many cases a few days after treatment. At most, just a small proportion of cells express proteins phenotypic of parenchymal cells. Expression of these proteins does not indicate true differentiation and neuronal or glial-cell function. After such a short period, even differentiated cells are highly unlikely to integrate truly into tissue and form complex connections, which improve function. Thus, tissue replacement as the mechanism by which MSC promote their beneficial effects is very unlikely. A far more reasonable explanation for the benefit is that MSC induce cerebral tissue to activate endogenous restorative effects of the brain. MSC can turn on reactions and interact with brain to activate restorative and possibly regenerative mechanisms.

MSC behave as small molecular factories, producing many different cytokines and trophic factors. MSC within cerebral tissue or within the microvasculature of injured brain are likely to express these factors, and the effect of the trophic factors on brain tissue is the mechanism that rapidly and effectively promotes restoration of function. Applicants have shown that MSC produce hepatocyte growth factor, VEGF, nerve growth factor (NGF), and brain-derived neurotrophic factor (BDNF), among many other trophic and growth factors. This variety of factors, and not the single bullet of a particular growth factor, facilitates the beneficial effect. A very important observation is that MSC when cultured under different ionic microenvironments respond to the cues by adjusting growth-factor expression. This finding suggests that cells within injured tissue express trophic and growth factors adjusted to the needs of the tissue. Different environments affect the secretion of these factors. Thus, the degree of tissue injury and the corresponding disruption of the ionic environment will dictate the secretion of trophic factors. Applicants have tested this hypothesis under several experimental conditions. Culture of MSC in tissues extracted from brains affected by stroke or injury significantly increase the secretion of trophic factors. The response secretions of MSC to the injured brain differ according to the time the tissue is extracted from the affected brain. These experiments were taken a step forward in the measurements of expression of growth factors in brain treated with MSC. Applicants used a quantitative sandwich ELISA, which measures by immunolabelling methods the expression of growth factors in brain.

Expression of trophic factors was significantly greater in MSC-treated animals than in non-treated animals subjected to stroke or trauma.

Given the assumption that MSC selectively enter injured brain and secrete growth and trophic factors in a tissue feedback loop, how do these factors alter the brain to promote therapeutic benefit? The operational hypothesis is that therapeutic benefit is induced by a set of events associated with brain plasticity; this process includes but is not limited to angiogenesis, neurogenesis, synaptogenesis, dendritic arborisation, and reduction of apoptosis within strategically important tissue in the boundary zone of the tissue.

VEGF and basic fibroblast growth factor are potent angiogenic agents. Applicants tested the effect of MSC or supernatant from MSC on the induction of angiogenesis. Measurements were made with an assay on human brain endothelial cells, in which supernatants from MSC were shown to induce the rapid formation of tubules, reflecting a structural and angiogenic process. The assay used in vivo was the classic avascular corneal assay. A surgical incision forms a pocket in the cornea, and a collagen wafer coated with MSC supernatant or MSC themselves are inserted in the pocket. Control conditions consisted of surgical incision and placement of the collagen wafer alone or placement of VEGF directly into the pocket. Applicants observed rapid and robust angiogenesis in the corneas treated with wafers loaded with MSC supernatant. Although most of the cells directly placed into the corneal incision diffuse away from the site, angiogenesis was evident. There was no angiogenesis in the control animals (FIG. 2). The induction of angiogenesis was more robust with the MSC supernatant than with the direct use of VEGF, which suggests that the supernatant is a highly effective source of angiogenic factors. Preliminary studies of angiogenesis induction by MSC treatment of brain tissue also suggest increased formation of new blood vessels (unpublished observation). Although the induction of angiogenesis does not directly translate into promotion of function, Applicants have previously shown that treatment of stroke with VEGF a day or more after stroke significantly improves functional recovery and increases angiogenesis.

Induction of neurogenesis by means of MSC can also contribute to functional improvement after stroke. An important site of neurogenesis is the area adjacent to the lateral ventricles—the subventricular zone. Neurogenesis is also found in the olfactory bulb and dentate gyrus of the rodent brain. Cerebral injury such as stroke amplifies the production of neurons within certain regions of the brain. Functional repair, particularly in the long term after a stroke, can be related to the production of new brain cells. Mechanisms that promote the production of these cells can improve recovery. Applicants tested the effects of treatment of stroke with MSC on induction of neurogenesis. A significant increase in cell numbers was measured in the subventricular zone after stroke. Many of these cells had markers of newly formed progenitor-like cells, as shown by the expression of specific molecular markers, such as TUJ-1. The cerebral tissue within the ipsilateral hemisphere also shows a massive increase of expression of the stem-cell marker, nestin, indicating the activation of cerebral tissue into a progenitor or developmental state. Histological analysis of the cerebral tissue transplanted with MSC also showed the presence of neurosphere rosettes within the ischaemic tissue. These rosettes of neuronal cells are similar to those found in the developing brain. The migration of these cell systems into the cerebral tissue can be guided by astrocytic-like projections emanating from the ventricular zone, again resembling events within the developing brain. Thus, the presence of bone-marrow cells seems to promote the rapid induction and migration of new cells from a primary source within the ventricular zone and the choroid plexus into the injured brain. These cells can contribute to functional repair, although the relation of the induction of neurogenesis and the migration of these cells to the restoration of function has not been directly tested.

The growth and trophic factors produced by MSC can affect synaptogenesis and increase dendritic arborisation in the injured and ischaemic brain. The direct effect of treatment of stroke with MSC on dendritic arborisation awaits further experiments. In preliminary experiments Applicants have shown increased expression of synaptophysin, a synaptic protein, within the boundary zone of the ischaemic lesion after stroke.

Gliosis can be an impediment to neurite outgrowth and arborisation after neural injury. The transforming growth factor•proteins are of major importance in wound healing and have been implicated in inhibition of scarring in skin and myocardium and the scarless wound repair observed in the fetus. Since MSC produce this growth factor, therapeutic benefit can also derive from the reduction of scarring and the subsequent improvement of synaptogenesis and dendritic arborisation.

In addition to cytokines and growth and trophic factors, MSC express factors associated with bone formation, such as osteoblast-specific factor 2 and bone morphogenetic protein 1. They also express the neural cell-adhesion molecule neuropilin and neurotrophic factors including NGF and BDNF. Recent studies have shown that bone morphogenetic proteins, sonic hedgehog, parathyroid hormone, and fibroblast growth factor eight have regulatory roles during differentiation of embryonic cells, by modifying mesodermal and neuroectodermal pathways. Whether the secretion by MSC of this cytokine cascade in injured brain contributes to functional benefit warrants careful consideration and further experiments.

The perilesional area is highly susceptible to apoptotic cell death. Apoptosis persists for months after stroke or brain trauma. The effects on recovery are unknown. Applicants have shown that treatment of stroke and brain trauma with MSC significantly reduces apoptosis within this area. The effect can be mediated by the production of growth factors, such as NGF, within the injured brain. Applicants speculate that the selective reduction of apoptosis within this region can sustain cerebral rewiring.

The mechanism by which brain remodelling, neurogenesis, and neuroprotective mechanisms evoke functional improvement after an injury is uncertain and an important topic of research. Whether all these events, which are amplified by treatment with MSC, actually contribute to improved outcome after stroke and trauma is under investigation.

At this time, specific events that foster restoration of neurological function cannot be isolated. Applicants speculate, however, that the process that promotes restoration of function is not single modification of tissue (eg, neurogenesis) but is most likely an interwoven set of events, angiogenesis, neurogenesis, synaptogenesis, and boundary reductions of scarring and apoptosis that contribute in a coupled if not synergistic manner to improve function. Although testing of this hypothesis and identification of the specific factors that contribute to improved neurological function is worthwhile, Applicants have limited ability to increase apoptosis selectively within the boundary zone, to reduce angiogenesis without affecting neurogenesis.

Injured cerebral tissue in many ways recapitulates ontogeny. After stroke or injury, cerebral tissue reverts to an earlier stage of development and thus becomes highly responsive to stimulation by cytokines and trophic and growth factors from the invading MSC. The MSC probably stimulate within the quasidevelopmental cerebral tissue structural and regenerative changes, including angiogenesis, vasculogenesis, neurogenesis, and dendritic arborisation. The primitive state of the tissue, which is highly sensitive to various stimulants and growth factors, rather than the primitive state of the MSC, primarily fosters a therapeutic response. The MSC can simply provide the resources required by the ontogenous cerebral tissue to stimulate cerebral remodelling. Applicants do not exclude the possibility that other cells or an orchestrated sequence of titrated infusions of cytokines and growth factors can stimulate the compromised brain cells to respond and to restore function. Similarly, Applicants cannot exclude the possibility that a subpopulation of MSC are stem-like or progenitor-like and can synergistically react with injured tissue. However, Applicants feel confident that the MSC within the brain do not replace tissue, and they do not differentiate into functioning neurons and supportive astrocytes, at least on the time scale in which Applicants see functional benefit. Primary benefit is obtained by activation of injured tissue to remodel and to compensate for injury. FIG. 3 illustrates the present understanding of the process by which MSC can be harvested and used to treat injured cerebral tissue.

Transplantation to Patients

Clearly, safety issues must be addressed before this form of cell therapy can be used in stroke patients. Although bone-marrow transplantation is a common procedure in cancer treatment and has been used as an adjunctive therapy in multiple sclerosis, phase I studies on safety in stroke are warranted. To date, in studies on nearly 2000 animals with stroke, Applicants have not detected any adverse effect of the therapy or indication of tumour formation. Should patients be treated with their own cells, HLA-matched cells, or a universal-donor population? The preclinical data so far suggest that treatment with donor cells is possible. However, preclinical and phase I clinical studies must be done to address this question. The preclinical and basic studies described in this review indicate that treatment of stroke with MSC can provide a viable and highly effective restorative therapy. Thus, clinical studies are warranted.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

TABLE
Differentiation of hMSCs Induced By TBI Tissue Extracts

| | | Neural-like cells |
|---|---|---|
| Groups | TBI Ext. (%) | (%) |
| Knockout DMEM | | 0 |
| Normal Brain Extracts | 20 | 3.16 ± 1.97 |
| | 40 | 2.08 ± 1.19 |
| TBI Extracts | 10 | 2.07 ± 0.49 |
| | 20 | 29.60 ± 16.89* |
| | 40 | 12.70 ± 8.49* |

hMSCs treated with TBI tissue extracts compare to control knockout DMEM with knockout serum replacement and normal brain extracts.
*P < 0.01

Strauss et al., “Germ line transmission of a yeast artificial chromosome spanning the murine a, (I) collagen locus”, Science, Vol. 259, pp. 1904-1907 (1993).
Gilboa, E, Eglitis, M A, Kantoff, P W, Anderson, W F: Transfer and expression of cloned genes using retroviral vectors. BioTechniques 4(6):504-512, 1986.
Cregg J M, Vedvick T S, Raschke W C: Recent Advances in the Expression of Foreign Genes in Pichia pastoris, Bio/Technology 11:905-910, 1993
Culver, 1998. Site-Directed recombination for repair of mutations in the human ADA gene. (Abstract) Antisense DNA & RNA based therapeutics, February, 1998, Coronado, Calif.
Huston et al, 1991 “Protein engineering of single-chain Fv analogs and fusion proteins” in Methods in Enzymology (J J Langone, ed.; Academic Press, New York, N.Y.) 203:46-88.
Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99.
Memaugh and Mernaugh, 1995 “An overview of phage-displayed recombinant antibodies” in Molecular Methods In Plant Pathology (R P Singh and U S Singh, eds.; CRC Press Inc., Boca Raton, Fla.) pp. 359-365.
AY H, AY I, KOROSHETZ W J, et al. (1999) Potential usefulness of basic fibroblast growth factor as a treatment for stroke. Cerebrovasc Dis 9:131-135.
AZIZI S A, STOKES D, AUGELLI B J, et al. (1998) Engraftment and migration of human bone marrow stromal cells implanted in the brains of albino rats—similarities to astrocyte grafts. Proc Natl Acad Sci USA 95:3908-3913.
BEREZOVSKAYA O, MAYSINGER D, and FEDOROFF S (1995) The hematopoietic cytokine, colony-stimulating factor 1, is also a growth factor in the CNS: congenital absence of CSF-1 in mice results in abnormal microglial response and increased neuron vulnerability to injury. Int J Dev Neurosci 13:285-299.
BJORKLUND A, and LINDVALL O (2000) Cell replacement therapies for central nervous system disorders. Nat Neurosci 3:537-544.
BULLOCK M R, LYETH B G, and MUIZELAAR J P (1999) Current status of neuroprotection trials for traumatic brain injury: lessons from animal models and clinical studies. Neurosurgery 45:207-217; discussion 217-220.
CANEVA L, SOLIGO D, CATTORETTI G, et al. (1995) Immuno-electron microscopy characterization of human bone marrow stromal cells with anti-NGFR antibodies. Blood Cells Mol Dis 21:73-85.
CHEN J, Li Y, WANG L, et al. (2001) Therapeutic benefit of intravenous administration of bone marrow stromal cells after cerebral ischemia in rats. Stroke 32:1005-1011.
CHOPP M, ZHANG X H, Li Y, et al. (2000) Spinal cord injury in rat: treatment with bone marrow stromal cell transplantation. Neuroreport 11:3001-3005.
DENG W, OBROCKA M, FISCHER I, et al. (2001) In vitro differentiation of human marrow stromal cells into early progenitors of neural cells by conditions that increase intracellular cyclic AMP. Biochem Biophys Res Commun 282:148-152.
DIXON C E, CLIFTON G L, LIGHTHALL J W, et al. (1991) A controlled cortical impact model of traumatic brain injury in the rat. J Neurosci Methods 39:253-262.
DORMADY S P, BASHAYAN O, DOUGHERTY R. et al. (2001) Immortalized-multipotential mesenchymal cells and the hematopoietic microenvironment. J Hematother Stem Cell Res 10:125-140.
GAGE F H (2000) Mammalian neural stem cells. Science 287:1433-1438.
HEFTI F (1986) Nerve growth factor promotes survival of septal cholinergic neurons after fimbrial transections. J Neurosci 6:2155-2162.
KOLIATSOS V E, CLATTERBUCK R E, WINSLOW J W, et al. (1993) Evidence that brain-derived neurotrophic factor is a trophic factor for motor neurons in vivo. Neuron 10:359-367.
KOPEN G C, PROCKOP D J, and PHINNEY D G (1999) Marrow stromal cells migrate throughout forebrain and cerebellum, and they differentiate into astrocytes after injection into neonatal mouse brains. Proc Natl Acad Sci USA 96:10711-10716.
KROMER L F (1987) Nerve growth factor treatment after brain injury prevents neuronal death. Science 235:214-216.
LABOUYRIE E, DUBUS P, GROPPI A, et al. (1999) Expression of neurotrophins and their receptors in human bone marrow. Am J Pathol 154:405-415.
LI Y, CHOPP M, CHEN J, et al. (2000) Intrastriatal transplantation of bone marrow nonhematopoietic cells improves functional recovery after stroke in adult mice. J Cereb Blood Flow Metab 20:1311-1319.
LU D, MAHMOOD A, WANG L, et al. (2001 a) Adult bone marrow stromal cells administered intravenously to rats after traumatic brain injury migrate into brain and improve neurological outcome. Neuroreport 12:559-563.
LU D, Li Y, WANG L, et al. (2001b) Intraarterial administration of marrow stromal cells in a rat model of traumatic brain injury. J Neurotrauma 18:813-819.
MAHMOOD A, LU D, Yl L, et al. (2001) Intracranial bone marrow transplantation after traumatic brain injury improving functional outcome in adult rats. J Neurosurg 94:589-595.
MAJUMDAR M K, THIEDE M A, MOSCA J D, et al. (1998) Phenotypic and functional comparison of cultures of marrow-derived mesenchymal stem cells (MSCs) and stromal cells. J Cell Physiol 176:57-66.
MAJUMDAR M K, THIEDE M A, HAYNESWORTH S E, et al. (2000) Human marrow-derived mesenchymal stem cells (mscs) express hematopoietic cytokines and support long-term hematopoiesis when differentiated toward stromal and osteogenic lineages. J Hematother Stem Cell Res 9:841-848.

MIZUNO S, MATSUMOTO K, KUROSAWA T, et al. (2000) Reciprocal balance of hepatocyte growth factor and transforming growth factor-beta 1 in renal fibrosis in mice. Kidney Int 57:937-948.
PAPAVASSILIOU E, GOGATE N, PROESCHOLDT M, et al. (1997) Vascular endothelial growth factor (vascular permeability factor) expression in injured rat brain. J Neurosci Res 49:451-460.
SANCHEZ-RAMOS J, SONG S, CARDOZO-PELAEZ F, et al. (2000) Adult bone marrow stromal cells differentiate into neural cells in vitro. Exp Neurol 164:247-256.
SONDELL M, and KANJE M (2001) Postnatal expression of VEGF and its receptor flk-1 in peripheral ganglia. Neuroreport 12:105-108.
SONDELL M, LUNDBORG G, and KANJE M (1999) Vascular endothelial growth factor has neurotrophic activity and stimulates axonal outgrowth, enhancing cell survival and Schwann cell proliferation in the peripheral nervous system. J Neurosci 0.19:5731-5740.
SUGIMORI H, SPELLER H, and FINKLESTEIN S P (2001) Intravenous basic fibroblast growth factor produces a persistent reduction in infarct volume following permanent focal ischemia in rats. Neurosci Lett 300:13-16.
TAKAIK, HARA J, MATSUMOTO K, et al. (1997) Hepatocyte growth factor is constitutively produced by human bone marrow stromal cells and indirectly promotes hematopoiesis. Blood 89:1560-1565.
WOODBURY D, SCHWARZ E J, PROCKOP D J, et al. (2000) Adult rat and human bone marrow stromal cells differentiate into neurons. J Neurosci Res 61:364-370.

ZHANG L, HIMI T, MORITA I, et al. (2000a) Hepatocyte growth factor protects cultured rat cerebellar granule neurons from apoptosis via the phosphatidylinositol-3 kinase/Akt pathway. J Neurosci Res 59:489-496.

ZHANG Z G, ZHANG L, JIANG Q, et al. (2000b) VEGF enhances angiogenesis and promotes blood-brain barrier leakage in the ischemic brain. J Clin Invest 106:829-838.

1. A therapeutic for use in inducing angiogenesis and vasculogenesis, said therapeutic comprising angiogenesis and vascologenesis inducing factors isolated from stem cells in conjunction with a pharmaceutically acceptable cell therapeutic, said therapeutic for inducing angiogenesis and vasculogenesis.

2. The therapeutic according to claim 1, wherein said angiogenesis and vasculogenesis inducing factors are selected from the group consisting essentially of angiogenic, trophic, and growth factors.

3. The therapeutic according to claim 1, wherein said cell therapeutic is a stem cell that is selected from the group consisting essentially of mesenchymal stem cells, stromal cells and precursors thereof, fibroblasts, reticulocytes, adipocytes, and endothelial cells.

4. A method of amplifying production of angiogenesis and vasculogenesis inducing factors secreted from stromal cells by exposing to and co-culturing stromal cells with a differentiation-inducing compound for increasing the production of the angiogenesis and vasculogenesis inducing factors.

5. The method according to claim 4, wherein said method includes exposing to and co-culturing stromal cells with a differentiation-inducing compound for increasing the production of the angiogenesis and vasculogenesis inducing factors., said differentiation-inducing compound being selected from the group consisting essentially of brain extract and calcium.

6. Angiogenesis and vasculogenesis inducing factors isolated and purified from stem cells for use as a therapy.

7. The angiogenesis and vasculogenesis inducing factors according to claim 6, wherein the angiogenesis and vasculogenesis inducing factors induce angiogenesis and vasculogenesis upon, administration to a patient in need of such treatment. 1 0

8. The angiogenesis and vasculogenesis inducing factors according to claim 6, wherein said angiogenesis and vasculogenesis inducing factors are factors secreted by stem cells when exposed to and co-cultured with a differentiation-inducing compound for increasing the production of desired factors.

9. The angiogenesis and vasculogenesis inducing factors according to claim 8, said differentiation-inducing compound being selected from the group consisting essentially of brain extract and calcium.

10. The angiogenesis and vasculogenesis inducing factors according to claim 9, wherein said brain extract is selected from the group consisting essentially of brain cells, cells obtained from the brain, and supernatant from stromal cells cultured with a medium.

11. A process for obtaining the angiogenesis and vasculogenesis inducing factors as set forth in claim 6, said process comprising the steps of:
isolating and purifying human mesenchymal stem cells from tissue prior to differentiation; and
culture expanding the mesenchymal stem cells for producing a tool for neurological and musculoskeletal therapy.

12. Isolated and culture expanded mesenchymal stem cells under the influence of a differentiation-inducing compound, said stem cells capable of differentiating and producing a desired cell phenotype needed for tissue repair.

13. The stem cells according to claim 12, said differentiation-inducing compound being selected from the group consisting essentially of brain 1 0 extract and calcium.

14. The stem cells according to claim 13, wherein said brain extract is selected from the group consisting essentially of brain cells, cells obtained from the brain, and supernatant from stromal cells cultured with a medium.

15. The stem cells according to claim 12 for use in redirecting reparative capacity of cells.

16. A therapy for inducing angiogenesis and vasculogenesis in a patient in need of said therapy by administering a therapeutic comprising angiogenesis and vasculogenesis inducing factors isolated from stem cells in conjunction with a pharmaceutically acceptable cell therapeutic, the therapeutic inducing angiogenesis and vagculogenesis in the patient.

17. The therapy according to claim 16, wherein said administering step includes administering the therapeutic in a manner selected from the group consisting essentially of subcutaneously, parenterally, intravenously, intraarterially, intramuscularly, intraperitoneally, intranasally, intrathecally, and via infusion techniques.

18. A method of inducing tissue repair by administering a therapeutic comprising angiogenesis and vasculogenesis inducing factors isolated from stem cells in conjunction with a pharmaceutically acceptable cell therapeutic, the therapeutic inducing tissue repair.

19. The method according to claim 18, wherein said administering step includes administering the therapeutic in a manner selected from the group consisting essentially of subcutaneously, parenterally, intravenously, intraarterially, intramuscularly, intraperitoneally, intranasally, intrathecally, and via infusion techniques.

20. A therapeutic for use in augmenting cellular function, said therapeutic comprising cellular function augmenting factors isolated from stem cells in conjunction with a pharmaceutically acceptable cell therapeutic, said therapeutic for augmenting cellular function.
